(19) United States
(12) Patent Application Publication
(10) Pub. No.: US 2025/0387114 A1
(43) Pub. Date: Dec. 25, 2025

(54) A DEVICE FOR TISSUE REPAIR, AND A METHOD OF REPAIRING A TISSUE USING THE DEVICE

(71) Applicants: TENDONPLUS MEDICAL PTE. LTD., Singapore (SG); ALEXANDRA HEALTH PTE. LTD., Singapore (SG)

(72) Inventors: Zai Hong Kua, Singapore (SG); Ahmed Khalil Khan Mohamed Hapiz Khan, Singapore (SG); Chung Hui Tan, Singapore (SG); Dong Hao Toon, Singapore (SG); Avshalom Shenhav, Misgav (IL); Zi An Wu, Singapore (SG)

(21) Appl. No.: 18/879,711

(22) PCT Filed: Jun. 30, 2023
(86) PCT No.: PCT/SG2023/050467

§ 371 Date: Dec. 27, 2024

(30) Foreign Application Priority Data
Jun. 30, 2022 (SG) 10202250346J

(51) Int. Cl. A61B 17/04; A61B 17/00

(52) U.S. Cl. CPC A61B 17/0401; A61B 2017/00477; A61B 2017/00526; A61B 2017/0409; A61B 2017/0412; A61B 2017/0414; A61B 2017/0427; A61B 2017/0456

(57) ABSTRACT

Disclosed is a device for tissue repair including a base plate having a first surface and a second surface opposing the first surface; a central shaft extending from the first surface of the base plate, the central shaft having an aperture configured to receive a suture; a plurality of anchor members extending from the first surface of the base plate, each of the plurality of anchor members having an end configured to penetrate the tissue, wherein each of the plurality of anchor members is spaced apart from the central shaft and spaced apart from another anchor member. The disclosure further includes a kit of parts of a tissue repair assembly including the device for tissue repair, an applicator and/or an installation device, and methods of repairing a tissue using the device.

### TECHNICAL FIELD

The present disclosure relates to a device for tissue repair, a kit of parts of a tissue repair assembly, and a method of repairing a tissue using the device.

### BACKGROUND

Tissue damage, tears or degeneration at the rotator cuff of a shoulder often requires surgical repair, including partial or complete reattachment of the torn or detached tissue to bone. The torn of detached tissue may be reattached by passing sutures through the tissue and pulling against the suture to allow the tissue to re-establish contact with bone, e.g. the greater tuberosity of the humerus bone, via bores drilled into the bone.

Before the tissue completely heals and reattaches to the bone, the suture bears the mechanical loads (tension) due to joint movements at the shoulder, resulting in localized shear stress at the tissue—suture interface and often causing the suture to cut though the soft tissue and cause the tissue to detach from the bone. For example, a common localized mechanical failure occurs when the suture used to attach torn tissue to bone, cuts through the reattached tissue under repeated tension or stress (“cheese-wiring effect”). In fact, there is a high re-tear rate for rotator cuff injuries (ranging from 20% to 90%), depending on the size of the original tear.

One solution for reducing the re-tear rate in rotator cuff injuries involves the use of complex suture configurations, by passing the suture through various locations of the tissue and securing the suture through additional knots. This involves forming many holes in the tissue which may weaken the mechanical integrity of the tissue, and it may be difficult and cumbersome for the clinician to perform the complex suture technique and to tie the various knots.

Another solution involves the use of flat sutures to distribute mechanical tension to the ends of the suture width. This procedure requires the use of suture penetrators or suture shuttles and requires additional steps during surgery. In addition, flat sutures in rotator cuff repair surgeries are susceptible to twisting.

Yet another solution involves the use of graft augmentation, which involves the application of a patch or a fascial graft to overlay the torn tissue, and securing the graft to the bone using sutures. Graft augmentation is a complex and expensive procedure that requires a pool of highly skilled and trained clinicians. Further, the use of fascial grafts may be unpleasant with respect to patient experience.

Accordingly, there exists a need to address at least one of the aforementioned issues.

### SUMMARY

The disclosure was conceptualized to provide an improved device for tissue repair, such as but not limited to rotator cuff repair. The device of the present disclosure may be positioned at the suture-tissue interface and may overlay the torn tissue. The device may thus minimize and reduce the occurrence of tissue detachment or re-tearing of the repaired rotator cuff, by reducing or minimizing direct contact between the suture and the tissue, and to redistribute mechanical tension or stress typically borne at the suture-tissue interface to various points on the tissue. For example, a point stress may be redistributed to various points on the tissue through the plurality of anchor members and in some embodiments, the central shaft, of the device. In addition, the plurality of anchor members of the device which are spaced apart from each other, and/or spaced apart from the central shaft, enables the individual adjustment of each anchor member and/or the central shaft of the device (which may be dependent on the size or severity of the tissue tear), and personalization of the device. Accordingly, the disclosure provides a personalized and customizable device which reduces and minimizes the recurrence of rotator cuff re-tearing or detachment. The device according to various embodiments of the disclosure is also compatible with existing suture configurations for rotator cuff repairs.

The device is particularly applicable for rotator cuff repairs, but can be used in the repair of soft tissue, for example in the repair of many other ligaments and tendons, or other connective tissue, such as in other joints in the body.

According to one aspect of the disclosure, a device for tissue repair is defined in claim 1. According to another aspect of the disclosure, a kit of parts of a tissue repair assembly including the device for tissue repair, an applicator and/or an installation device is defined in claims 35 to 45. According to yet another aspect of the disclosure, methods of repairing a tissue is defined in claims 46 to 53.

The dependent claims define some embodiments associated with the device, the kit of parts of the tissue repair assembly, and the method of repairing a tissue.

### BRIEF DESCRIPTION OF THE DRAWINGS

The disclosure will be better understood with reference to the detailed description when considered in conjunction with the non-limiting examples and the accompanying drawings, in which:

FIGS. 1A to 1C show various views of an embodiment of the device 10A. FIGS. 1A and 1B show the perspective views, and FIG. 1C shows the top view of the device 10A;

FIGS. 2A to 2D show various views of another embodiment of the device 10B. FIGS. 2A and 2B show the perspective views, FIG. 2C shows the top view of base plate 110, and FIG. 2D show the top view of the device 10B;

FIGS. 3A to 3D show various views of another embodiment of the device 10C. FIGS. 3A and 3B show the perspective views, FIG. 3C shows the top view of the base plate 110, and FIG. 3D shows the top views of the device 10C;

FIGS. 4A to 4D show various views of another embodiment of the device 10D. FIGS. 4A and 4B show the perspective views, FIG. 4C shows the top view of the base plate 110, and FIG. 4D shows the top view of the device 10D;

FIGS. 5A to 5D show various views of another embodiment of the device 10E. FIGS. 5A and 5B show the perspective views, FIG. 5C shows the top view of the base plate 110, and FIG. 5D shows the top view of the device 10E;

FIGS. 6A to 6D show various views of another embodiment of the device 10F. FIGS. 6A and 6B show the perspective views, FIG. 6C shows the top view of the base plate 110, and FIG. 6D shows the top view of the device 10F;

FIGS. 7A to 7D show various views of another embodiment of the device 10G. FIG. 7A shows the perspective view, FIG. 7B shows the side view, and FIG. 7C shows the top view of the base plate 110 of the device 10G. FIG. 7D shows an enlarged view of an anchor member 130 of the device 10G;

FIGS. 8A to 8C show various views of another embodiment of the device 10H. FIGS. 8A and 8B show the perspective views, and FIG. 8C shows the cross-sectional view of the device 10H;

FIGS. 9A to 9D show various views of another embodiment of the device 10I. FIGS. 9A and 9B shows the perspective views, FIG. 9C shows the top view of the base plate 110, and FIG. 9D shows the top view of the device 10I;

FIGS. 10A to 10B show various views of another embodiment of the device 10J. FIG. 10A shows the perspective view, and FIG. 10B shows the cross-sectional view of device 10J;

FIGS. 11A to 11D show various views of another embodiment of the device 10K. FIGS. 11A and 11B shows the perspective views, and FIG. 11C shows the side view of the device 10I. FIG. 11D shows an enlarged view of an anchor member 130 of the device 10K;

FIGS. 12A to 12C show various views of yet another embodiment of the device 10L. FIG. 12A shows the perspective view, FIG. 12B shows the cross-sectional view, and FIG. 12C shows the side view of the device 10L;

FIGS. 13A to 13E show various view of another embodiment of the device 10M. FIG. 13A shows a perspective view of the disassembled parts of the device 10M, FIGS. 13B and 13D show the perspective views of various embodiments of the assembled device 10M, and FIGS. 13C and 13E shows the cross-sectional views of the views shown in FIGS. 13B and 13D, respectively;

FIGS. 14A to 14D show various view of another embodiment of the device 10N. FIGS. 14A and 14B show perspective views of the device 10N, FIG. 14C shows the top view of the base plate 110, and FIG. 14D shows the top view of the device 10N;

FIGS. 15A to 15D show various view of another embodiment of the device 10O. FIGS. 15A and 15B show perspective views of the device 10O, FIG. 15C shows the top view of the base plate 110, and FIG. 15D shows a side view of the device 10O;

FIGS. 16A to 16D show various view of another embodiment of the device 10P. FIGS. 16A and 16B show perspective views of the device 10P, and FIGS. 16C and 16D show perspective views of the base plate 110 in an unfolded configuration and a folded configuration, respectively;

FIG. 17 shows the perspective view of another embodiment of the device 10Q;

FIG. 18A to 18C show various views of another embodiment of the device 10R. FIGS. 18A and 18B show the perspective views, and FIG. 18C shows the side view of the device 10R;

FIGS. 19A to 19C show various views of another embodiment of the device 10S. FIGS. 19A and 19B show the perspective views, and FIG. 19C shows the side view of the device 10S;

FIGS. 20A to 20C show various views of another embodiment of the device 10T. FIGS. 20A and 20B show the perspective views, and FIG. 20C shows the side view of the device 10T;

FIGS. 21A to 21D show various views of another embodiment of the device 10U. FIGS. 21A and 21B show the perspective views, FIG. 21C shows the side view of the device 10U, and FIG. 21D shows the top view of the base plate 110;

FIGS. 22A to 22C show various views of another embodiment of the device 10V. FIGS. 22A and 22B show the perspective views, and FIG. 22C shows the side view of the device 10V;

FIGS. 23A to 23C show various views of another embodiment of the device 10W. FIGS. 23A and 23B show the perspective views, and FIG. 23C shows the side view of the device 10W;

FIGS. 24A to 24D show various views of another embodiment of the device 10X. FIGS. 24A and 24B show the perspective views, and FIG. 24C shows the side view of the device 10X;

FIGS. 25A to 25C show various views of another embodiment of the device 10Y. FIGS. 25A and 25B show the perspective top and bottoms views, respectively, and FIG. 25C shows the bottom view of device 10Y;

FIGS. 26A to 26C show various views of another embodiment of device 10Z. FIG. 26A shows a perspective view and inset (i.) shows an exploded view of a sickle-head tip 2602 and the third fluke 2604 of device 10Z. FIG. 26B shows a side view, and FIG. 26C shows a front view of device 10Z;

FIGS. 27A to 27C show various views of another embodiment of device 10AB. FIG. 27A shows a perspective view of device 10AB, FIG. 27B shows a platform 2820 of an applicator 2800 having an anvil 2822 configured to crimp the end 132 of the elongate portion 136 of device 10AB, and FIG. 27C shows side views of the device AB, (i.) before and (ii.) after entry into a tissue for repair;

FIGS. 28A and 28B show an example of a kit of parts of a tissue repair assembly 20A comprising the device 10A as described with reference to FIGS. 1A to 1C;

FIGS. 29A and 29B show an example of a kit of parts of a tissue repair assembly 20B comprising device 10V as described with reference to FIGS. 22A to 22C, and an applicator 2800 including an embodiment of an effector 2900;

FIGS. 30A and 30B show an example of a kit of parts of a tissue repair assembly 20C comprising the base plate 110 of device 10Y as described with reference to FIGS. 25A to 25C, and an applicator 2800 including an embodiment of an effector 3000;

FIGS. 31A and 31B show an example of a kit of parts of a tissue repair assembly 20D comprising the base plate 110 of device 10Y as described with reference to FIGS. 25A to 25C, and an applicator 2800 including an embodiment of an effector 3100;

FIG. 32 shows another embodiment of an exemplary kit of parts of a tissue repair assembly 20E comprising the device 10Q as described with reference to FIG. 17;

FIGS. 33A to 33C show another embodiment of an exemplary kit of parts of a tissue repair assembly 20F comprising the device 10I as described with reference to FIGS. 9A to 9D;

FIGS. 34A and 34B show another embodiment of an exemplary kit of parts of a tissue repair assembly 30 comprising an installation device 3400 and an applicator 2800;

FIG. 35 shows an exemplary flowchart of a method 3500 of attaching a tissue repair device to an applicator;

FIGS. 36A to 361 show exemplary schematics 3600 of method 3500 for attaching an exemplary tissue repair device to the applicator 2800, and the removal of the installation device 3400 from the applicator 2800;

FIG. 37 shows an embodiment of a method 3700 of repairing a tissue, according to another aspect of the disclosure;

FIG. 38 shows another embodiment of method 3800 of repairing a tissue, according to another aspect of the disclosure;

FIG. 39 shows another embodiment of method 3800 of repairing a tissue, according to another aspect of the disclosure; and

FIGS. 40A to 40C shows schematic illustrations showing the conventional repair of torn tissue with and without the device 4000.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the disclosure. Other embodiments may be utilized and structural, and logical changes may be made without departing from the scope of the disclosure. The various embodiments are not necessarily mutually exclusive, as some embodiments can be combined with one or more other embodiments to form new embodiments.

The disclosure illustratively described herein may suitably be practiced in the absence of any element or elements, limitation or limitations, not specifically disclosed herein. Thus, for example, the terms “comprising”, “including,” “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically described in exemplary embodiments and optional features, modification and variation of the disclosure embodied herein may be resorted to by those skilled in the art.

In the context of various embodiments, the articles “a”, “an” and “the” as used with regard to a feature or element include a reference to one or more of the features or elements. As used herein, the term “and/or” includes any and all combinations of one or more of the associated listed items.

Throughout the description, the term “tissue” as used herein, refers to connective tissue proper or soft tissue, including but not limited to tendons and ligaments. The term “tissue” is also contemplated to include cartilage. Within the context of rotator cuff repairs, the term “tissue” may refer to the supraspinatus tendon.

Throughout the description, the term “repair” as used herein, refers broadly to surgical means for the repair of the tissue, for example, surgery to repair a torn rotator cuff by reattaching the rotator cuff to the humerus bone. The term “repair” thus includes arthroscopy, open surgery, or a combination of both for rotator cuff repairs.

Throughout the description, the term “perimeter” as used herein, refers broadly to an edge, e.g. end, border or boundary of a base plate. In some embodiments, the term may refer to a circumference of a base plate.

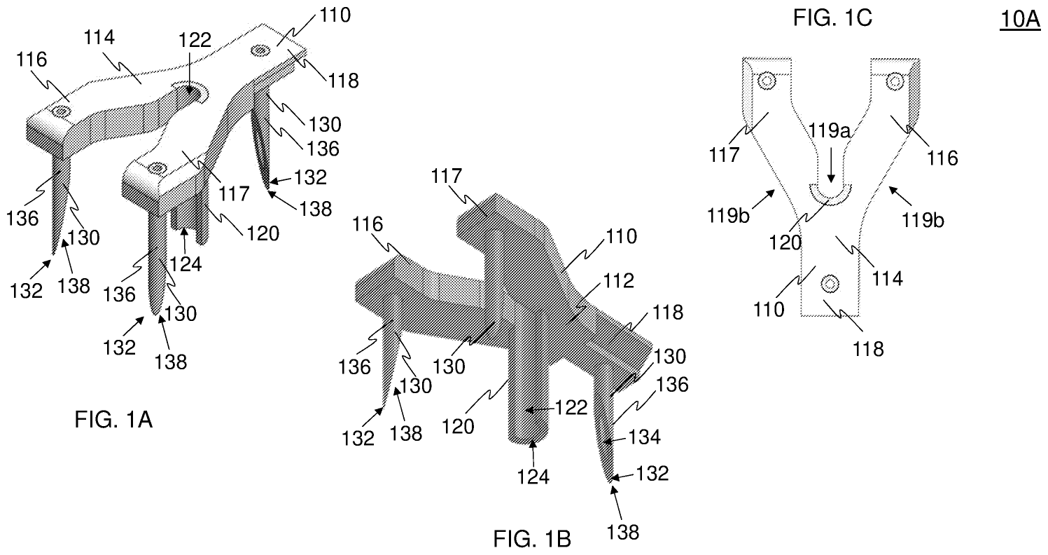

According to one aspect of the disclosure, there is provided a device for tissue repair, the various embodiments of which are discussed with reference to FIGS. 1A to 27C.

FIGS. 1A to 1C show various views of an embodiment of the device 10A. FIGS. 1A and 1B show the perspective views, and FIG. 1C shows the top view of the device 10A. Referring to FIGS. 1A to 1C, the device 10A for tissue repair includes a base plate 110 having a first surface 112 and a second surface 114 opposing the first surface 112. In some embodiments, the first surface 112 may refer to the bottom surface of the device 10A, which may be configured to be in contact with the repaired tissue. The second surface 114 may refer to the top surface of the device 10A, and may work in tandem, or may contact an applicator designed to receive and deliver the device 10A to the tissue for repair, e.g. an injury site.

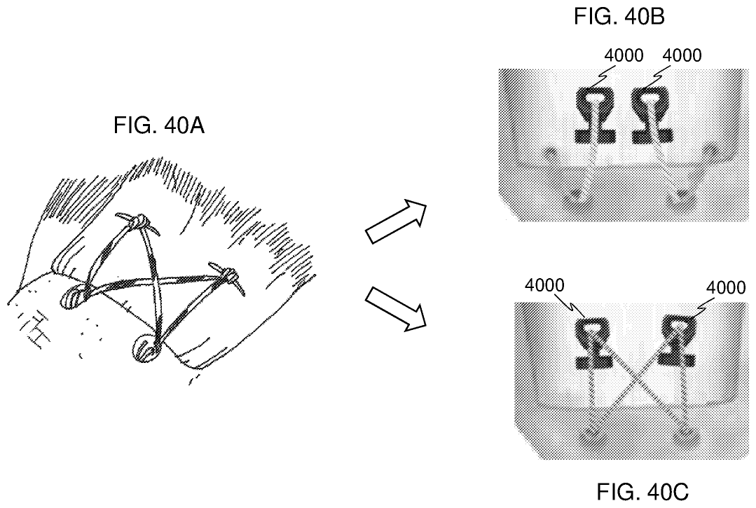

The base plate 110 includes an aperture 122 arranged on a portion of the base plate 110, the aperture 122 configured to receive a suture, e.g. a surgical suture such as but not limited wire or thread-like sutures or flat sutures. In various embodiments, the aperture 122 may be configured to receive one or more sutures (see FIG. 40). The suture may be positioned in the aperture 122 and along a longitudinal axis of the device 10A. In device 10A, the base plate 110 may include a first segment 116, a second segment 117 and a third segment 118, which may be connected to each other at the portion of the base plate 110 having the aperture 112. As shown in FIGS. 1A to 1C, the base plate 110 may be formed in a Y-shape, e.g. alphabetic character “Y”, where the first 116 and second 117 segments may extend from the aperture 122 of the base plate 110, and along a lateral axis of the device 10A. The first 116 and second 117 may be identical to each other, and spaced apart from each other by a first angle 119a. In some embodiments, the first 116 and second 117 segments may have a same thickness and length, and the first angle 119a may be an acute angle. The third segment 118 may also extend from the central shaft 120 along the lateral axis of the device 10A. The third segment 118 may differ to the first 116 and second 117 segments. In some embodiments, the third segment 118 may have a different length and/or thickness from each of the first 116 and second 117 segments. For example, the third segment 118 may have a non-uniform thickness, and a thickness at a portion from which an anchor member 130 extends from the third segment 118 may be less than a thickness of another portion of the third segment 118. The reduced thickness may be a contact or retaining profile to provide easy attachment to an applicator designed to receive and deliver the device 10A. In some embodiments, the third segment 118 may be spaced apart from each of the first 116 and second 117 segments by a second angle 119b, which differs to the first angle 119a. For example, the second angle 119b may be an obtuse angle. Alternatively, it is contemplated that the first angle 119a may be an obtuse angle, and the second angle 119b an acute angle. It is also contemplated that each of the first 116, second 117 and third 118 segments may be different from each other, and may be separated by different angles.

The device 10A may include a central shaft 120 extending from the first surface 112 of the base plate 110, the central shaft 120 having the aperture 122 arranged on the portion of the base plate 110. In other words, the central shaft 120 may include the aperture 122. In some embodiments, the central shaft 120 may have an end 124 configured to penetrate the tissue. For example, the end 124 of the central shaft 120 may be sharp to pierce and penetrate the tissue. In the embodiment as shown in FIGS. 1A to 1C, the central shaft 120 may include a partial circle, for example, half a circle, or a semi-circle, e.g. “open-ended” shape, which may provide ease of threading and aligning the suture into the aperture 122.

The device 10A further includes a plurality of anchor members 130 extending from the first surface 112 of the base plate 110, each of the plurality of anchor members 130 having an end 132 configured to penetrate the tissue. For example, the ends 132 of the anchor members 130 may be sharp to pierce and penetrate the tissue. Each of the plurality of anchor members 130 is spaced apart from the central shaft 120, and spaced apart from another anchor member 130. For example, each of the plurality of anchor members 130 may be separate and independent to the central shaft 120, and may not be in contact with, or connected to the central shaft 120. In the same manner, each of the plurality of anchor members 130 are separate and independent to each other 130, and may not be in contact with, or connected to another anchor member 130. In the embodiment as shown in FIGS. 1A to 1C, the device 10A may include three anchor members 130, each of them being separate to each other, and separate to the central shaft 120. It is contemplated that the device 10A may include two, four, five or more anchor members 130, each of them being separate to each other, and separate to the central shaft 120.

Accordingly, the device 10A of the present disclosure may provide for the individual adjustment of each anchor member 130 separate from another anchor member(s) 130, and/or separate from the central shaft 120. For example, a length of an anchor member 130 may differ, e.g. shorter or longer, to another anchor member 130, and/or the central shaft 120. As a further example, the length of each anchor member 130 in device 10A may be different to each other, and may be different to the central shaft 120. In another example, an anchor member 130 may extend perpendicular to the base plate 110, and another anchor member 130 may extend at an angle, e.g. acute or obtuse, to the base plate 110. That is, each of the plurality of anchor members 130 may be adjusted and calibrated according to the tissue for repair, e.g. size and severity of tear in tissue, to provide a personalized, tailor-made device for tissue repair. For example, as tissue thickness may not be consistent along the site of the tear, longer anchor members 130 may be used for thicker tissue points, and shorter anchor members 130 for thinner tissue points.

In the embodiment shown in FIGS. 1A to 1C, the plurality of anchor members 130 of device 10A may be spaced apart from a perimeter of the base plate 110. For example, each of the plurality of anchor members 130 may be spaced apart from the edge of the base plate 110, for example, the edges or ends of the first 116, second 117 and third 118 segments. Each of the plurality of anchor members 130 may include an elongate portion 136 which extends perpendicular from the base plate 110. Alternatively, the elongate portion 136 may extend at an angle, for example, an acute or obtuse angle, from the base plate 110. The elongate portion 136 may include a beveled tip 138 which forms the end 132 configured to penetrate the tissue. For example, the tip of the elongate portion 136 may be slanted and sharp to pierce and penetrate the tissue. In some embodiments, the elongate portion 136 may include a lumen 134 configured to receive the suture, and may provide additional points where the suture may pass to anchor and embed the device 10A on the tissue for repair. In various embodiments, the lumen 134 may be configured to receive more than one suture.

It is further contemplated that agents such as therapeutic agents and/or reinforcing agents may be administered into the tissue via the lumen 134 and/or the central shaft 120. For example, reinforcing or bonding agents such as growth factors or cross-linking agents may be injected into the tissue via the lumen 134 and/or central shaft 120, to strengthen the tissue and/or to promote wound healing. Alternatively, or in addition, the agent may be administered as a liquid bead on the end 124, 132 of the central shaft 120 and/or the plurality of anchor members 130, or may be pre-coated on the central shaft 120 and/or the plurality of anchors members 130 before the device 10A is delivered to the tissue.

According to various embodiments of the device of the disclosure, a surface area of the central shaft 120 may be greater than a surface area of the suture, along the longitudinal axis of the device 10A, e.g. along the length of the central shaft 120. As such, the central shaft 120 may have a larger bearing area as compared to the suture, thereby reducing peak stress on the tendon.

In the embodiment shown with reference to FIGS. 1A to 1C, a length of the central shaft 120 may be the same as a length of each of the plurality of anchor members 130. In some other embodiments, the length of the central shaft 120 may be longer or shorter than each of the plurality of anchor members 130. For example, the length of the central shaft 120 may be longer that an anchor member 130, and may be shorter than another anchor member 130, according to the requirements of the tissue that is to be repaired using the device 10A.

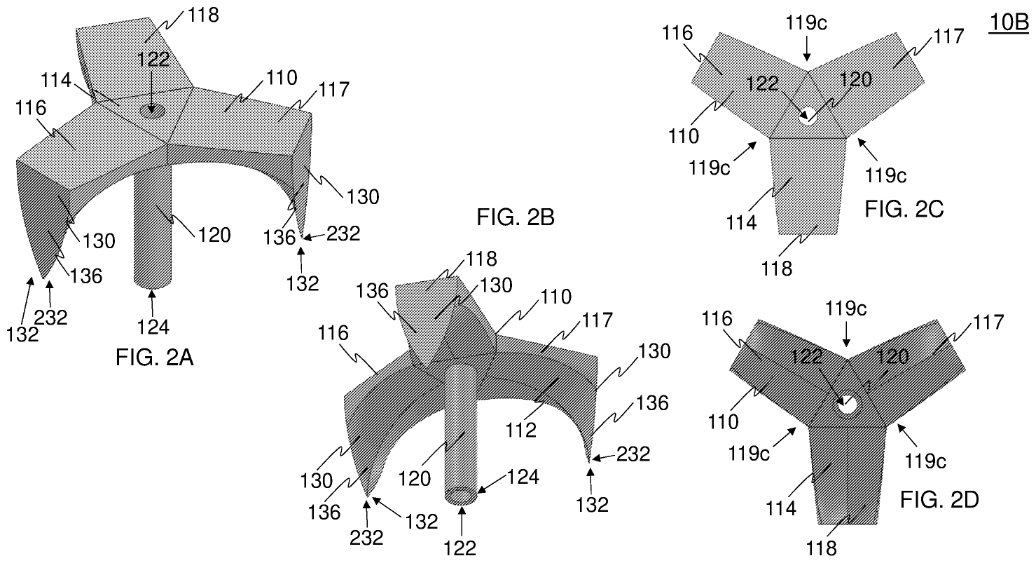

FIGS. 2A to 2D show various views of another embodiment of the device 10B.

FIGS. 2A and 2B show the perspective views, FIG. 2C shows the top view of base plate 110, and FIG. 2D show the top view of the device 10B. The device 10B as shown in FIGS. 2A to 2D may share similarities as device 10A, specifically, in that the device 10B includes a base plate 110 having a first surface 112 and a second surface 114 extending from the first surface 112; a central shaft 120 extending from the first surface 112 of the base plate and having an aperture 122 configured to receive a suture; and a plurality of anchor members 130 extending from the first surface 112 of the base plate 110, each of the plurality of anchor members 130 having an end 132 configured to penetrate tissue, wherein each of the plurality of anchor members 130 is spaced apart from the central shaft 120 and spaced apart from another anchor member 130. The description of device 10B will thus focus on the variations of device 10B.

Referring to FIGS. 2A to 2D, the base plate 110 of the device 10B may include a first segment 116, a second segment 117 and a third segment 118, wherein the first 116, second 117 and third 118 segments may be connected to each other at the position from which the central shaft 120 extends from the first surface 114 of the base plate 110. In device 10B, the first 116, second 117 and third 118 segments may be identical, and may have for example, a same thickness and length. Each of the first 116, second 117 and third 118 segments may be equally spaced apart by a third angle 119c, and the third angle 119c may be an acute angle. In some embodiments, the base plate 110 may be an isotoxal polygon, e.g. isotoxal star, and each of the first 116, second 117 and third 118 segments may be spaced apart from each other by the third angle 119c, which may be equal to 120°.

The device 10B may include a central shaft 120 which may be a complete or full circle having the aperture 122 for receiving a suture. For example, a suture may be threaded through the aperture 122 of the central shaft 120. In device 10B, each of the plurality of anchor members 130 may each include an elongate portion 136 arranged along a perimeter, specifically, the edges of the first 116, second 117 and third 118 segments of the base plate 110. The anchor members 130 may extend perpendicular to the base plate 110 and may have a width which tapers towards the end 132 of the anchor member 130. For example, a width of the elongate portion 136 anchor member 130 proximal to the base plate 110 may be greater than a width proximal to the end 132 of the anchor member 130. Alternatively, the plurality of anchor members 130 may extend at an angle to the base plate 110, and the plurality of anchor members 130 may each extend at a different angle to another anchor member 130. In some embodiments, the end 132 of the plurality of anchor members 130 may include a pointed tip 232 which may be sharp for piercing and penetrating the tissue. This may allow a user, e.g. clinician, to easily insert the device 10B into the tissue for repair.

It is contemplated that an agent, such as a reinforcing and/or therapeutic agent may be administered to the tissue through the central shaft 120 and/or the plurality of anchor members 130. For example, the agent may be administered via injection through the central shaft 120, or administered as a liquid bead positioned at the end 124 of the central shaft 120 and/or the pointed tip 232 of the plurality of anchor members 130. Alternatively, or in addition, the central shaft 120 and/or the plurality of anchor members 130 may be pre-coated with said agent.

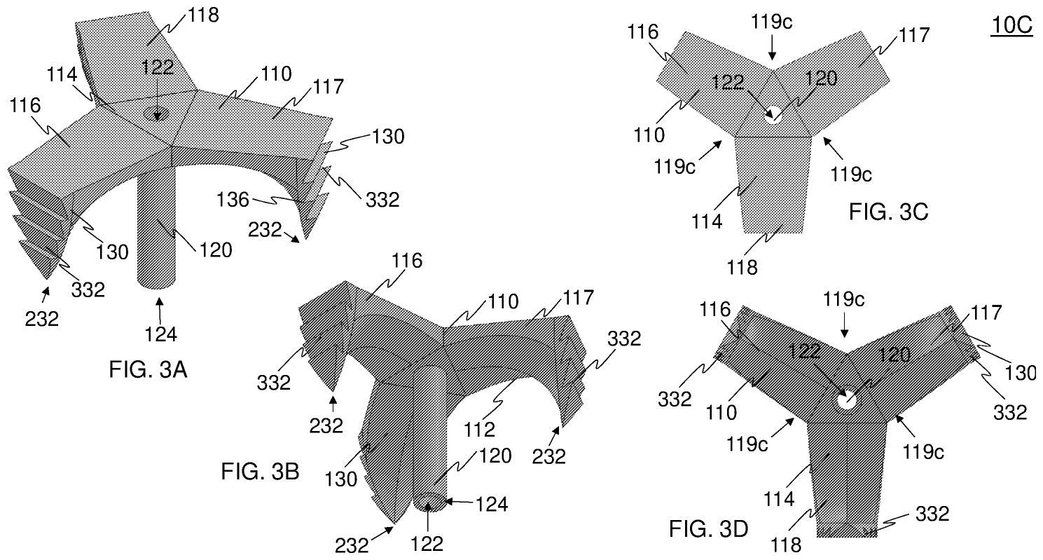

FIGS. 3A to 3D show various views of another embodiment of the device 10C. FIGS. 3A and 3B show the perspective views, FIG. 3C shows the top view of the base plate 110, and FIG. 3D shows the top views of the device 10C. The device 10C as discussed with reference to FIGS. 3A to 3D may be based on device 10B described with reference to FIGS. 2A to 2D and repeated descriptions will be omitted. For example, the base plate 110 and the central shaft 120 may be the same as that described with reference to device 10B.

As shown in FIGS. 3A to 3D, the plurality of anchor members 130 may each include an elongate portion 136 extending perpendicular to the first surface 112 of each of the first 116, second 117, and third 118 segments of the base plate 110, and having a pointed 232 and sharp tip which forms the end 132 configured to penetrate the tissue. Alternatively, the elongate portions 136 may extend at an angle to the first surface 112 of the base plate 110, and each elongate portion 136 of the plurality of anchor members 130 may extend at different angles to each other. Similar to device 10B, the elongate portion 136 may have a width that tapers towards the pointed end 232 of the plurality of anchor members 130.

In some embodiments, each of the elongate portions 136 of the plurality of anchor members 130 may further include a set of teeth 332 arranged along a length of the elongate portions 136, such that the surface of the elongate portion 136 may rough and serrated (unlike device 10B where the surface of the elongate portion 136 may be smooth). In other words, the length of the elongate portion 136 may be uneven and may have jagged peaks arranged along the length of the elongate portion 136. The jagged peaks of the set of teeth 332 may provide additional contact interfaces between the plurality of anchor members 130 of the device 10C and the tissue and may thus provide stronger anchorage of the device 10C to the tissue. In addition, the jagged peaks of the set of teeth 332 may provide a larger pre-coating surface area when an agent is applied to the plurality of anchor members 130 before delivery of the device 10C into the tissue. It is also contemplated that the length of the plurality of anchor members 130 may include barbs.

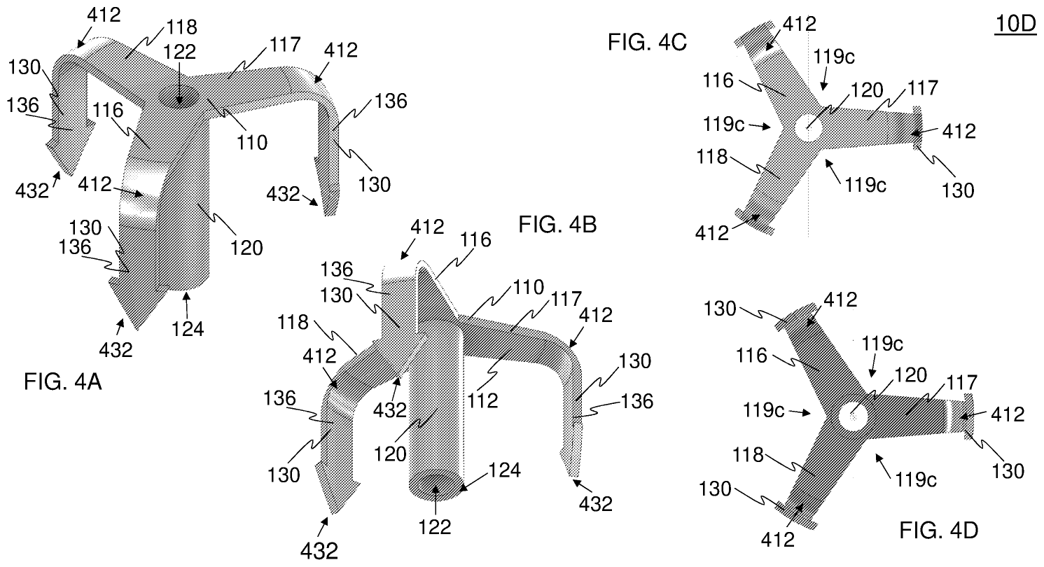

FIGS. 4A to 4D show various views of another embodiment of the device 10D. FIGS. 4A and 4B show the perspective views, FIG. 4C shows the top view of the base plate 110, and FIG. 4D shows the top view of the device 10D. The device 10D may be similar to device 10B and 10C described with reference to FIGS. 2A to 3D and repeated descriptions will be omitted. For example, the base plate 110 and the central shaft 120 may be the same as that described with reference to device 10B and 10C, wherein the base plate 110 may include a first 116, second 117 and third segment 118, which are connected to each other at a position from which the central shaft 120 extends from the first surface 112 of the base plate 110. The first 116, second 117 and third 118 segments may be identical to each other and may each be spaced apart by the third angle 119c. The central shaft 120 may include a full circle with an aperture 122 configured to receive the suture.

Referring to FIGS. 4A to 4D, the junction from which the plurality of anchor members 130 extends from the base plate 110 may be rounded 412. For instance, the edges of the base plate 110 may be rounded 412, such that the plurality of anchor members 130 which extends from the edges, e.g. perimeter, of the first 116, second 117 and third 118 segments of the base plate 110, may not extend perpendicular to the base plate 110. The rounded 412 portion of the base plate 110 may provide for a better fit at corners where the device 10D and the tissue are in contact with each.

The plurality of anchor members 130 may each include an elongate portion 136 having an arrowhead tip 432 which forms the end 132 of configured to penetrate the tissue. For example, the arrowhead tip 432 may be sharp to pierce the tissue. The arrowhead tip 432 may provide additional reinforcement to anchor and embed the device 10D into the tissue, by providing additional contact interfaces between the edges of the arrowhead tip 432 and the tissue. The edges of the arrowhead tip 432 may act as a “hook or buckle” to secure the anchor member 130 into the tissue and to strengthen the anchorage provided by the device 10D.

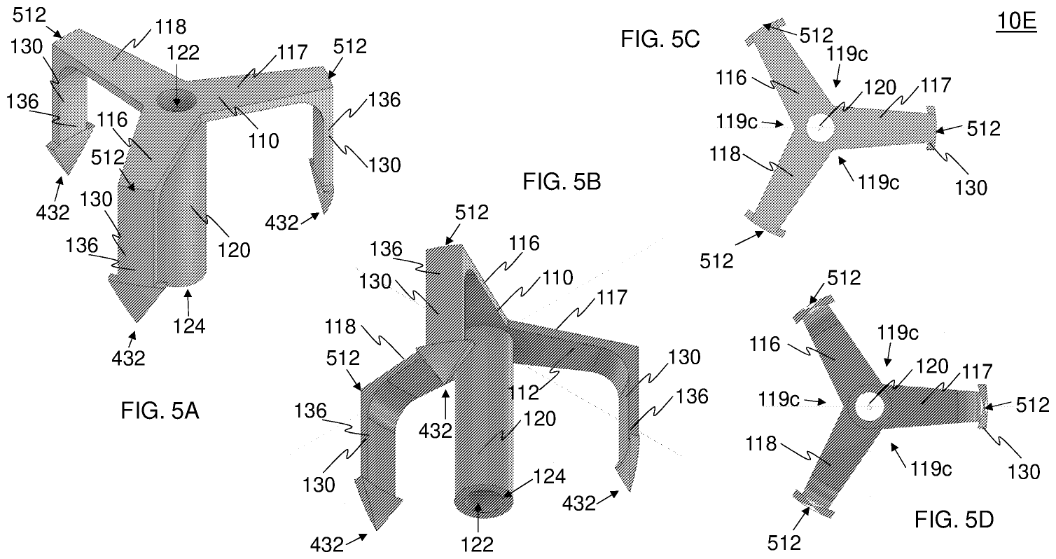

FIGS. 5A to 5D show various views of another embodiment of the device 10E. FIGS. 5A and 5B show the perspective views, FIG. 5C shows the top view of the base plate 110, and FIG. 5D shows the top view of the device 10E. The device 10E may be based on device 10D described with reference to FIGS. 4A to 4D and repeated descriptions will be omitted. As may be seen in FIGS. 5A to 5D, the base plate 110 including the first 116, second 117 and third 118 segments, and the central shaft 120 may be the same as device 10D.

In the embodiment as shown in FIGS. 5A to 5D, the plurality of anchor members 130 may be arranged to extend perpendicular along the perimeter of the base plate 110. In other words, the junction where each of the plurality of anchor member 130 extends from the ends or edges of each of the first 116, second 117, and third 118 segments forms a corner edge 512. This provides additional stiffness to the anchor members 130, and reduces the risk that the corner edge 512 deforms, and/or the elongate portion 136 of the anchor members 130 bend upon insertion into tissue.

The plurality of anchor members 130 may each include an elongate portion 136 having the arrowhead tip 432 which may be sharp to form the end 132 configured to penetrate the tissue, in a similar manner to device 10D described with reference to FIGS. 4A to 4D. The arrowhead tip 432 may provide additional reinforcement to anchor and embed the device 10E into the tissue, by providing additional contact interfaces between the edges of the arrowhead tip 432 and the tissue. The edges of the arrowhead tip 432 may act as a “hook or buckle” to secure the anchor member 130 into the tissue and to strengthen the anchorage provided by the device 10E.

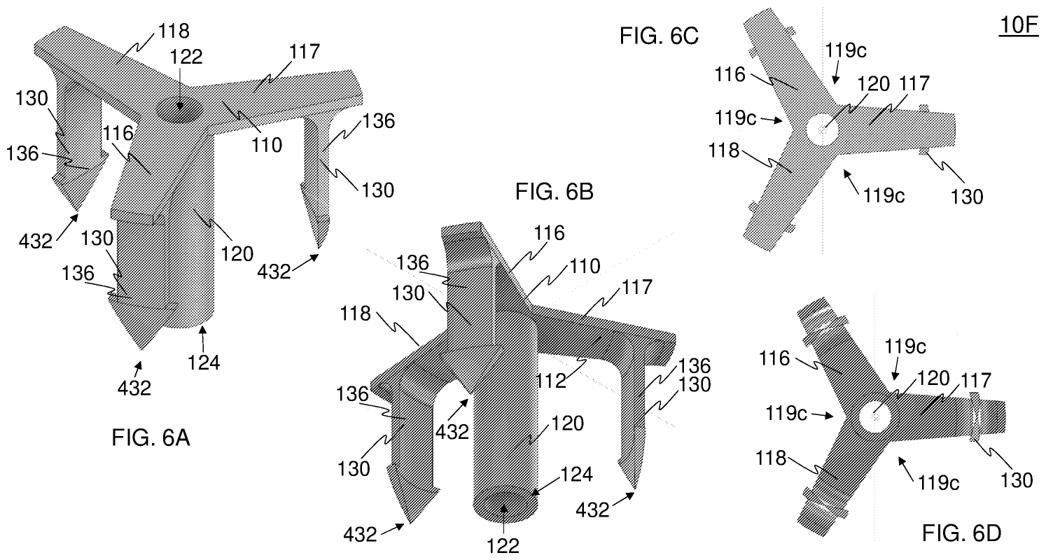

FIGS. 6A to 6D show various views of another embodiment of the device 10F. FIGS. 6A and 6B show the perspective views, FIG. 6C shows the top view of the base plate 110, and FIG. 6D shows the top view of the device 10F. The device 10F may be based on device 10D and 10E described with reference to FIGS. 4A to 5D and repeated descriptions will be omitted. As may be seen in FIGS. 6A to 6D, the base plate 110 including the first 116, second 117 and third 118 segments, and the central shaft 120 may be the same as device 10D and 10E.

Referring to FIGS. 6A to 6D, the plurality of anchor members 130 may extend perpendicular to the first 116, second 117 and third 118 segments of the base plate 110. In the embodiment as shown with respect to device 10F, the plurality of anchor members 130 may be spaced apart from the perimeter of the base plate 110. As such, there is increased bearing area between the tissue and the device 10F, which prevents the anchor members 130 from sinking too deep into the tissue when excessive force is used during insertion of the device 10F into tissue. Further, the anchor members 130 which are spaced apart from the perimeter of the base plate 110 may have improved stiffness, which may reduce the risk of bending at the interface at which the elongate portion 136 extends from the first 116, second 117 or third 118 segments. Each of the plurality of anchor members 130 may include an elongate portion 136 having the arrowhead tip 432 which forms the end 132 configured to penetrate the tissue, in a similar manner to device 10D and 10E, and repeated descriptions will be omitted.

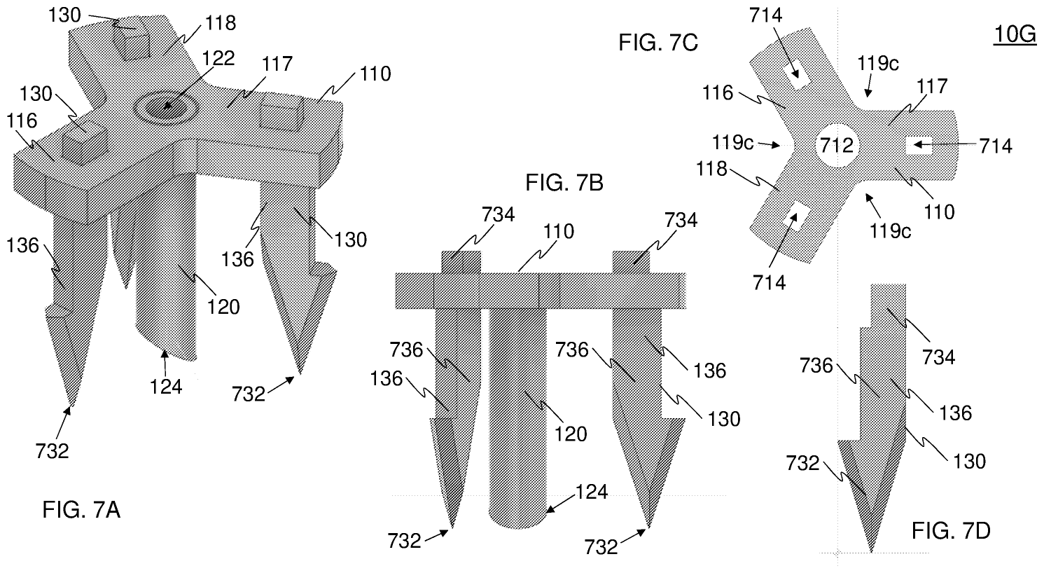

FIGS. 7A to 7D show various views of another embodiment of the device 10G. FIG. 7A shows the perspective view, FIG. 7B shows the side view, and FIG. 7C shows the top view of the base plate 110 of the device 10G. FIG. 7D shows an enlarged view of an anchor member 130 of the device 10G. The device 10G may share similarities to exemplary embodiments of device 10D, 10E and 10F described with reference to FIGS. 4A to 6D. For example, the base plate 110 may include a first 116, second 117 and third segment 118, which are connected to each other at a position from which the central shaft 120 extends from the base plate 110. The first 116, second 117 and third 118 segments may be identical to each other and may each be spaced apart by the third angle 119c.

Referring to FIGS. 7A to 7D, the central shaft 120 may include a full circle with an aperture 122 configured to receive the suture. In some embodiments, the end 124 of the central shaft 124 may have an inclined profile and may be beveled. In accordance with various embodiments of the device, the end 124 of the central shaft 124 may be sharp to pierce and penetrate the tissue.

In some embodiments, the base plate 110 may include a shaft through-hole 712, which may be shaped and dimensioned to receive the central shaft 120, for example, the top portion of the central shaft 120. The shaft through-hole 712 may be positioned at the center of the base plate 110. The central shaft 120 may be therefore be detachable to the base plate 110.

In some embodiments, the plurality of anchor members 130 are configured to be detachable to the base plate 110. For example, the base plate 110 may include a plurality of through-holes 714 which may be shaped and dimensioned to receive the plurality of anchor members 130 (see FIG. 7C). The plurality of through-holes 714 may be positioned along each of the first 116, second 117 and third 118 segments. In a further example, the plurality of though-holes 714 may be shaped and dimensioned to receive a first part 734 of the elongate portion 136 of the plurality of anchor members 130. Although FIG. 7C depicts one through-hole 714 positioned along each of the first 116, second 117 and third 118 segments, it is contemplated that more than one through-hole 714 may be positioned along each of the first 116, second 117 and third 118 segments of the base plate 110. Accordingly, each of the first 116, second 117 and third 118 segments may receive more than one anchor member 130.

FIG. 7D shows an enlarged view of an exemplary embodiment of an anchor member 130. In some embodiments, the plurality of anchor members 130 of the device 10G may include an elongate portion 136 having an arrowhead tip 732. The elongate portion 136 of the anchor member 130 may include a first part 734 and a second part 736 positioned adjacent to the first part 734 and proximal to the arrowhead tip 732. The first part 734 may have a first thickness and the second part 736 may have a second thickness greater than the first thickness. In other words, the anchor members 130 may be designed to have an increasing thickness towards the arrowhead tip 732. As shown in FIG. 7D, the interfaces between the first 734 and second 736 parts, and the second 736 part and the arrowhead tip 732 may be uneven. For example, the interfaces may include a jagged edge. The uneven interfaces may provide additional contact interfaces between the tissue and the device 10G, which may act as a “hook or buckle” to secure the anchor member 130 into the tissue and to strengthen the anchorage provided by the device 10G. Accordingly, the suture-tissue interface may be strengthened due to the device 10G, and the suture and device 10G may not detach from the tissue upon repeated tension and stress.

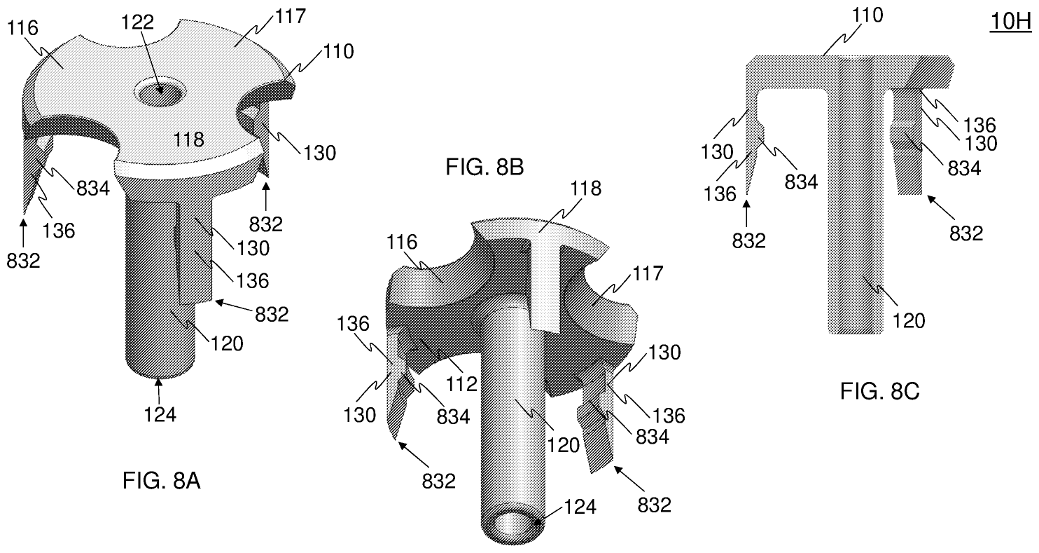

FIGS. 8A to 8C show various views of another embodiment of the device 10H. FIGS. 8A and 8B show the perspective views, and FIG. 8C shows the cross-sectional view of the device 10H.

Referring to FIGS. 8A to 8C, the base plate 110 may include a first 116, second 117 and third segment 118, which are connected to each other at a position from which the central shaft 120 extends from the base plate 110. The first 116, second 117 and third 118 segments may be identical to each other and may be separated, e.g. spaced apart from each other by an arc along the perimeter, e.g. circumference of the base plate 110. The device 10H may also include a central shaft 120 having an aperture 122 configured to receive a suture, and an end 124 which may be flat and sharp to penetrate the tissue.

The plurality of anchor members 130 may extend from the perimeter of the base plate 110. For example, the plurality of anchor members 130 may extend from the edges of each of the first 116, second 117 and third 118 segments. As shown in FIGS. 8A to 8C, the plurality of anchor members 130 may each include an elongate portion 136 which extends perpendicular to the base plate 110. In some embodiments, the elongate portion 136 may include a projection 834 arranged along the length of the elongate portion 136. For example, the projection 834 may protrude towards the central shaft 120. In addition, the plurality of anchor members 130 including the elongate portion 136 may have a flat end 832 that may be sharp to pierce and penetrate the tissue. The projection 834 may act as a “hook or buckle” to secure the plurality of anchor members 130 into the tissue and to strengthen the anchorage provided by the device 10H. Accordingly, the suture-tissue interface may be strengthened due to the device 10H, and the suture and device 10H may not detach from the tissue upon repeated tension and stress. In addition, the plurality of anchor members 130 including the projection 834 may provide a larger pre-coating surface area when an agent is applied to the plurality of anchor members 130 before delivery of the device 10H into the tissue.

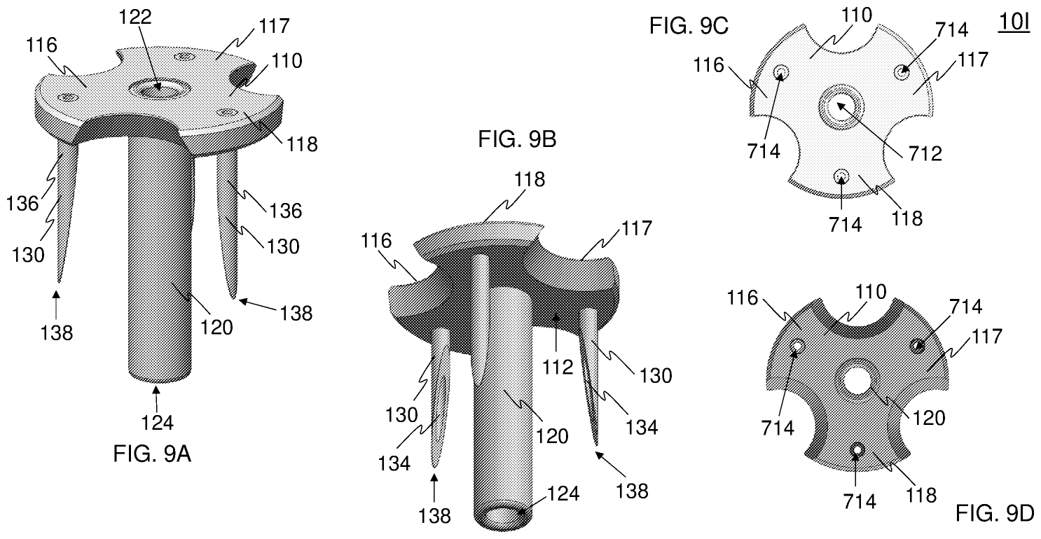

FIGS. 9A to 9D show various views of another embodiment of the device 10I. FIGS. 9A and 9B shows the perspective views, FIG. 9C shows the top view of the base plate 110, and FIG. 9D shows the top view of the device 10I. The device 10I is based on device 10H described with reference to FIGS. 8A to 8C and repeated description will be omitted. For example, the shape of the base plate 110, and the central shaft 120 may be the same as device 10H in FIGS. 8A to 8C.

Referring to FIGS. 9A to 9D, the central shaft 120 may be detachable to the base plate 110. For example, the base plate 110 may include a shaft through-hole 712, which is configured to receive the central shaft 120, for example, the top portion of the central shaft 120. In addition, the base plate 110 may include a plurality of through-holes 714 configured to receive the plurality of anchor members 130, which may also be detachable from the base plate 110. For example, the plurality of through-holes 714 may be dimensioned to receive the top portion of each of the plurality of anchor members 130, for attaching the plurality of anchor members 130 to the base plate 110.

The plurality of anchor members 130 may be spaced apart from the perimeter, e.g. circumference of the base plate 110. The plurality of anchor members 130 may each include an elongate portion 136 extending perpendicular to the first surface 112 of the base plate 110, and may further include a beveled tip 138 which forms the end 132 configured to penetrate the tissue, and a lumen 134 which may be configured to receive a suture.

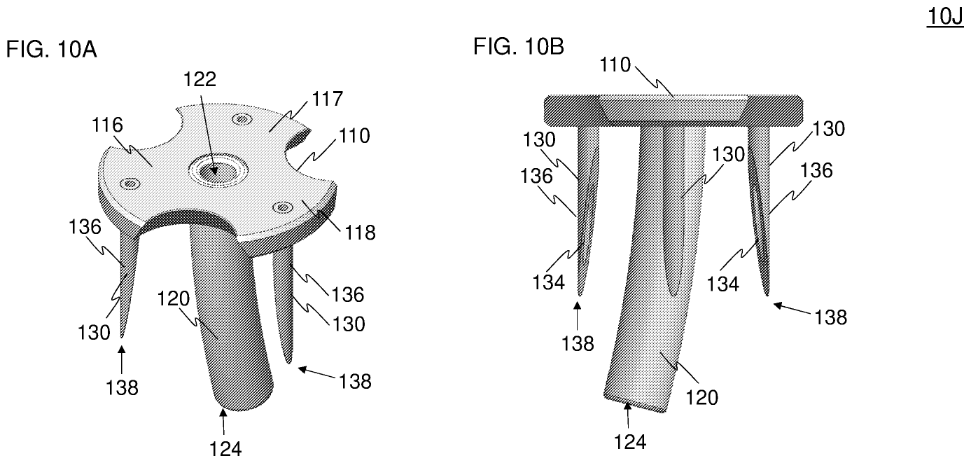

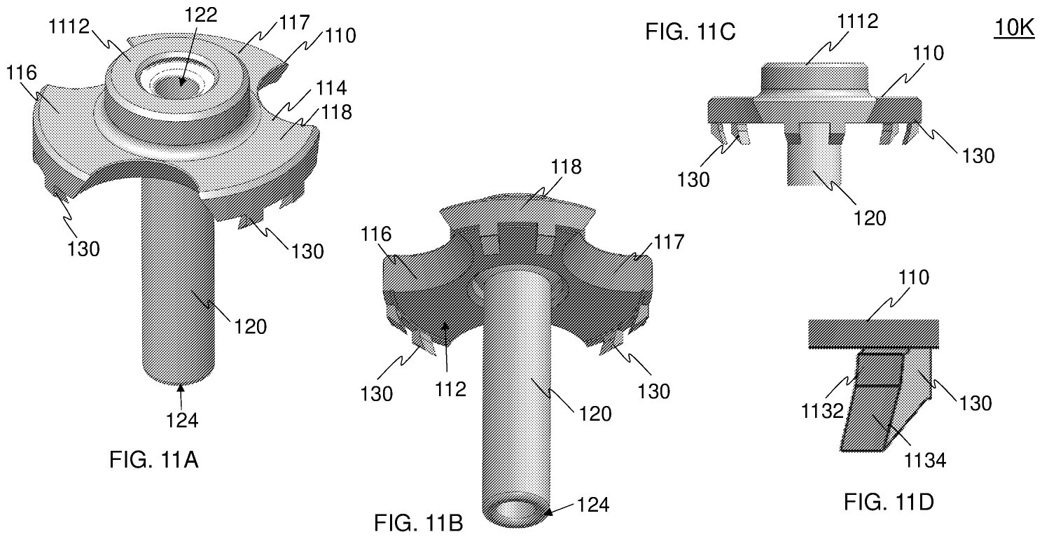

FIGS. 10A to 10B show various views of another embodiment of the device 10J.

FIG. 10A shows the perspective view, and FIG. 10B shows the cross-sectional view of device 10J. The device 10J is based on device 10H and 10I described with reference to FIGS. 8A to 9D and repeated description will be omitted. For example, the shape of the base plate 110 may be the same as device 10H in FIGS. 8A to 8C, and the plurality of anchor members 130 may be the same as device 10I in FIGS. 9A to 9D.

Referring to FIGS. 10A and 10B, the central shaft 120 of the device 10J may be pivotable with respect to a portion, for instance, the central portion, of the base plate 110. For example, the central shaft 120 may extend from the first surface 112 at an angle to the base plate 110, and the angle may include an acute or obtuse angle. In some embodiments, a mechanism such as a pivotable interface may be provided at the interface of the base plate 110 and the central shaft 120. Accordingly, the central shaft 120 may be positioned by a user, e.g. a clinician depending on the size and severity of the tissue tear, to provide a personalized device 10J for a patient.

FIGS. 11A to 11D show various views of another embodiment of the device 10K. FIGS. 11A and 11B shows the perspective views, and FIG. 11C shows the side view of the device 10I. FIG. 11D shows an enlarged view of an anchor member 130 of the device 10K.

Referring to FIGS. 11A to 11C, the base plate 110 may be configured to have the first 116, second 117 and third 118 segments, each segment separated by an arc of the base plate 110. Each of the first 116, second 117 and third 118 segments may be connected to each other at a position from which the central shaft 120 extends from the base plate 110. In some embodiments, the central shaft 120 may extend perpendicular to the base plate 110, and may include a full circle having an end 124 that may be sharp to penetrate the tissue.

As shown in FIGS. 11A to 11C, the base plate 110 may also include a protrusion 1112 extending from the second surface 114 of the base plate 110. For example, the protrusion 1112 may be shaped to surround the circumference of the central shaft 120 and may provide an opening leading to the aperture 122 of the central shaft 120, to allow a suture to be threaded through the aperture 122 of the central shaft 120. The protrusion 1112 may provide means to mount the device 10K to an applicator (described in accordance with another aspect of the disclosure below), for delivery of said device 10H to the torn tissue, e.g. an injury site.

FIG. 11D shows an enlarged view of an exemplary embodiment of an anchor member 130. Each of the plurality of anchor members 130 of the device 10K may include a first portion 1132 extending from the first surface 112 of each of the first 116, second 117, and third 118 segments of the base plate 110. In some embodiments, the first portion 1132 may extend perpendicular to the base plate 110. Each of the plurality of anchor members 130 may also include a second portion 1134 extending from an end of the first portion 1132. The second portion 1134 may extend at an angle to the first portion 1132 and may extend towards the central shaft 120, e.g. towards the central axis of the base plate 110, of the device 10K. The plurality of anchor members 130, each having the first 1132 and second 1134 portions, may act as a “hook or buckle” to secure the plurality of anchor members 130 into the tissue to strengthen the grip and anchorage provided by the device 10K. Accordingly, the suture-tissue interface may be strengthened due to the device 10K, and the suture and device 10K may not detach from the tissue upon repeated tension and stress.

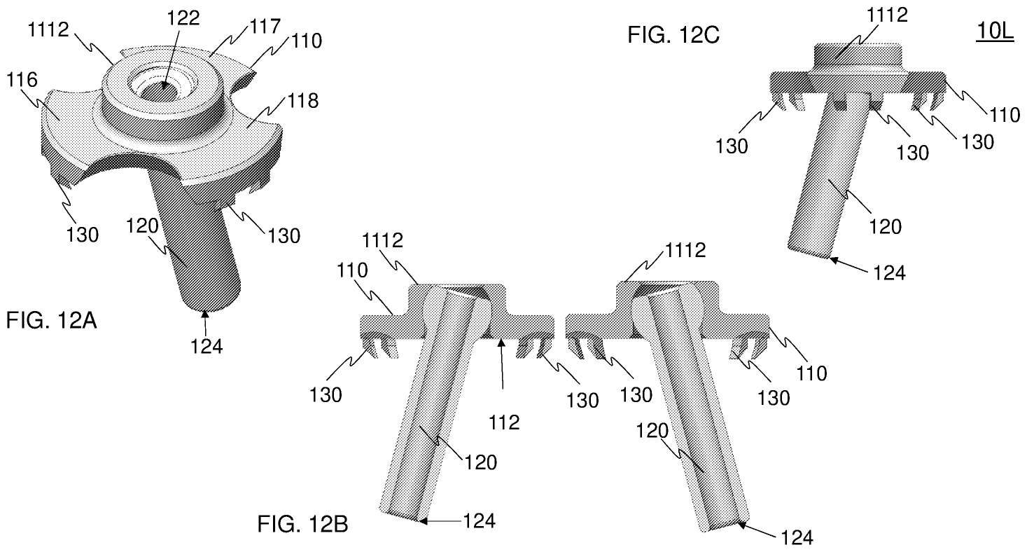

FIGS. 12A to 12C show various views of yet another embodiment of the device 10L. FIG. 12A shows the perspective view, FIG. 12B shows the cross-sectional view, and FIG. 12C shows the side view of the device 10L. Device 10L may be based on device 10K described with reference to FIGS. 11A to 11D and repeated descriptions will be omitted. For example, the base plate 110 may include the protrusion 1112 extending from the second surface 114 of the base plate 110, and each of the plurality of anchor members 130 may include the first portion 1132 and second portion 1134 extending from the end of the first portion 1132 and towards the central shaft 120.

In the embodiments as shown in FIGS. 12A to 12C, the central shaft 120 of device 10L may by pivotable with respect to a portion, for example, the central portion of the base plate 110, and may extend from the first surface 112 at an acute or obtuse angle to the base plate 110. As shown in FIG. 12B, the central shaft 120 may be either be positioned towards the right or the left with respect to the longitudinal axis of the device 10L. In some embodiments, a mechanism such as a pivotable interface may be provided at the interface of the base plate 110 and the central shaft 120. Accordingly, the central shaft 120 may be positioned by a user, e.g. a clinician depending on the size and severity of the tissue tear, to provide a personalized device 10L for a patient.

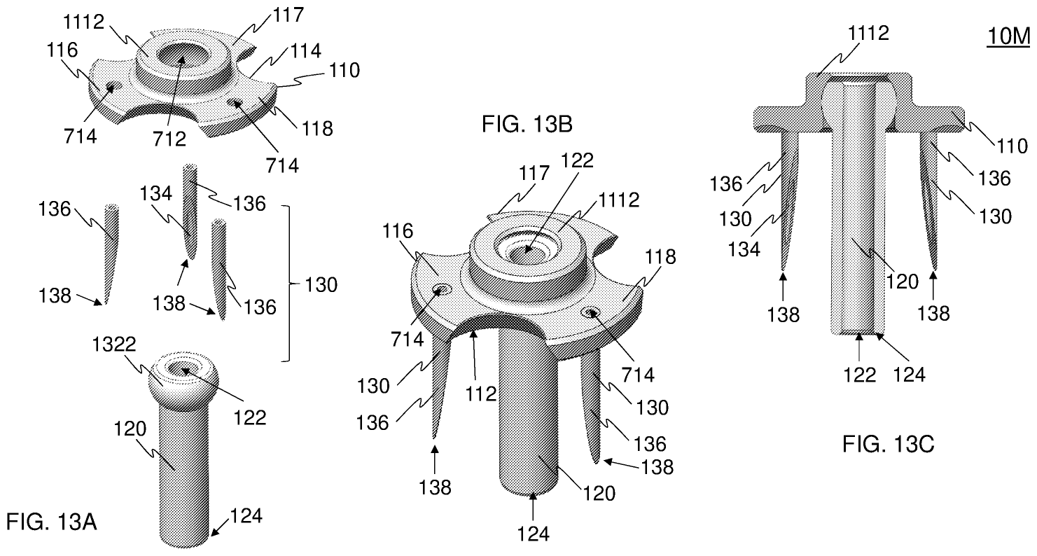

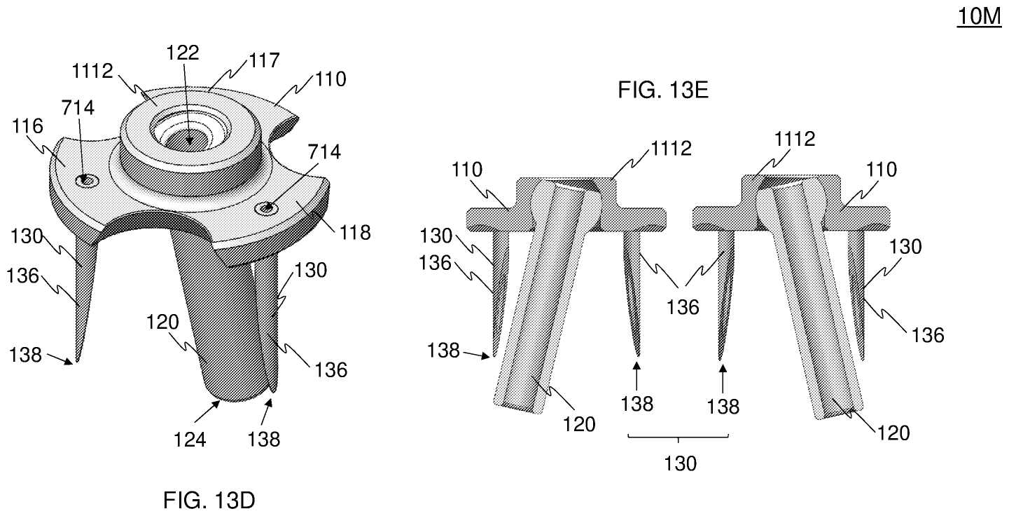

FIGS. 13A to 13E show various view of another embodiment of the device 10M. FIG. 13A shows a perspective view of the disassembled parts of the device 10M, FIGS. 13B and 13D show the perspective views of various embodiments of the assembled device 10M, and FIGS. 13C and 13E shows the cross-sectional views of the views shown in FIGS. 13B and 13D, respectively.

Referring to FIGS. 13A to 13C, the device 10M may include a base plate 110 having the first 116, second 117 and third 118 segments, the segments connected to each other at the position from which the central shaft 120 is configured to extend from the base plate 110. The base plate 110 may further include a protrusion 1112 extending from the second surface 114 of the base plate 110. The base plate 110 may be the base plate 110 as described with reference to device 10K, 10L in FIGS. 11A to 12C, and will not be described in detail with reference to device 10M.

As shown in FIG. 13A, the central shaft 120 may include a bolt 1322 positioned at an end of the central shaft 120. For example, the bolt 1322 may be positioned at an end which is opposite to the end 124 that may be sharp to penetrate the tissue.

The base plate 110 may include the shaft through-hole 712 which is configured to receive the central shaft 120. In some embodiments, the shaft through-hole 712 may be dimensioned according to the size, e.g. diameter of the bolt 1322 of the central shaft 120 and the bolt 1322 may fit into the shaft through-hole 712, for attaching the central shaft 120 to the base plate 110. The base plate 110 may further include a plurality of through-holes 714 arranged on the base plate 110, and configured to receive the plurality of anchor members 130. The plurality of through-holes 714 may be configured according to the type of each of the plurality of anchor members 130 that is to be fitted onto the base plate 110. For example, the plurality of through-holes 714 may be round and may have a circumference which matches a diameter of a round elongate portion 136 of the plurality of anchor members 130. As a further example, the plurality of through-holes 714 may be rectangular to receive the first portion 1132 of each of the plurality of anchor members 130, as shown in FIGS. 11A to 12C).

The plurality of anchor members 130 may each include an elongate portion 136 including a beveled tip 138 forming the end 132 configured to penetrate the tissue, and a lumen 134 configured to receive a suture and/or an agent which may be administered into the tissue via the lumen 134. The plurality of anchor members 130 may be the same as that described with reference to device 10A, 10I, 10J in FIGS. 1A to 1C, and 9A to 10B. In some embodiments, an end, for example, the end opposing the beveled tip 138 of each of the plurality of anchor members 130 may be attached to a respective through-hole 714 arranged on the base plate 110, to form the plurality of anchor members 130 of the device 10M.

The disassembled device 10M comprising parts of the base plate 110, the central shaft 120, and the plurality of anchor members 130 may be easily packaged as a flat pack and delivered to a user, e.g. clinician. The various parts of the device 10M may also allow the clinician to select a suitable base plate 110 type, central shaft 120 type and/or the type of each of the plurality of anchor members 130, depending on the severity and size of the tissue tear, to provide a personalized device 10M. It is also contemplated that each of the plurality of anchor members 130 may differ to each other. For example, a device 10M may include an anchor member 130 having an elongate portion 136 with a beveled tip 138, an elongate portion 136 having a pointed tip 232, and/or an anchor member 130 having a first portion 1132 extending from the base plate 110 and a second portion 1134 extending from the end of the first portion 1132.

FIGS. 13D and 13E show another embodiment of device 10M, wherein the central shaft 120 may be positioned to be pivotable with respect to a portion, e.g. central portion, of the base plate 110. For example, the shaft through-hole 712 may be configured to allow the bolt 1322 of the central shaft 120 to be pivotable, such that it may be positioned at an angle to the base plate 110.

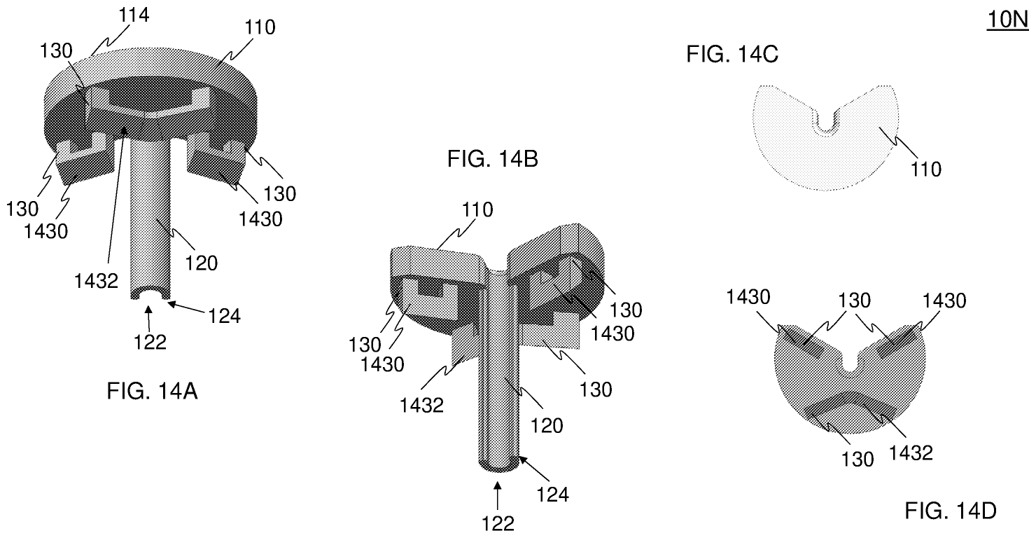

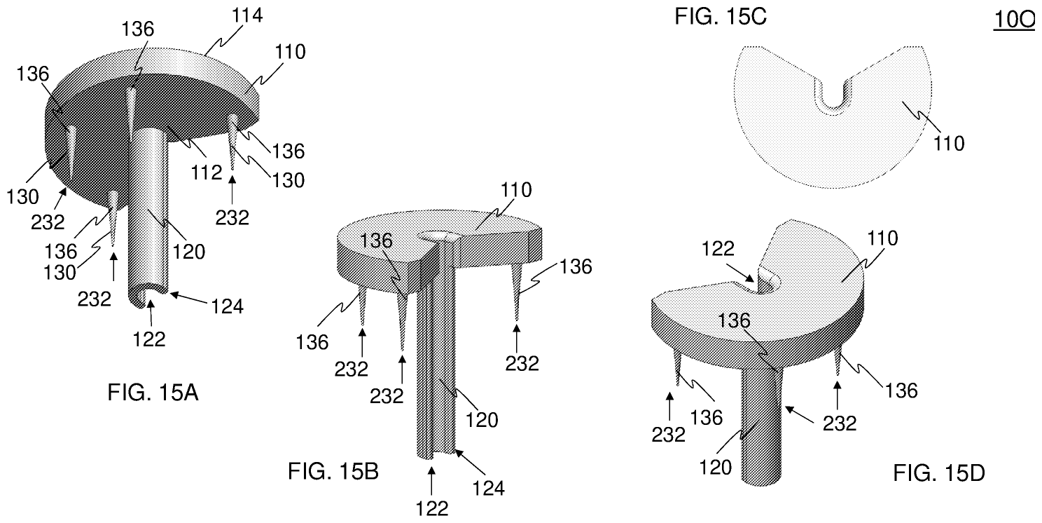

FIGS. 14A to 14D show various view of another embodiment of the device 10N. FIGS. 14A and 14B show perspective views of the device 10N, FIG. 14C shows the top view of the base plate 110, and FIG. 14D shows the top view of the device 10N. Device 10N may share similarities with any of device 10A to 10M described with reference to FIGS. 1A to 13E, specifically, in that the device 10N comprises a base plate 110 having a first 112 and a second 114 surface opposing the first surface 112; a central shaft 120 extending from the first surface 112 of the base plate 110 and having an aperture 122 configured to receive a suture; a plurality of anchor members 130 extending from the first surface 112 of the base plate, each of the plurality of anchor members 130 having an end 132 to penetrate the tissue, wherein each of the plurality of anchor members 130 are spaced apart from the central shaft 120 and spaced apart from another anchor member 130.

Referring to FIGS. 14A to 14D, the base plate 110 may include a partial circle. In some embodiments, the base plate 110 may be three quarters of a full circle. The central shaft 120 may include a partial circle, for example, half a circle or a semi-circle extending from the base plate 110. The partial circle shape of the central shaft 120 may allow the user, e.g. clinician to easily align and/or thread a suture through the aperture 122 of the central shaft 120.

Each of the plurality of anchor members 130 and the central shaft 120 may extend from the first surface 112 of the base plate 110, and may be separate and spaced apart from each other, so as to allow individual adjustment and calibration of each anchor member 130 and the central shaft 120 for providing a personalized device for tissue repair. As shown in FIGS. 14A to 14D, the plurality of anchor members 130 may include a rectangular frame 1430 extending from the base plate 110, and the length of the rectangular frame 1430 may be sharp to form the end 132 configured to penetrate the tissue. In some embodiments, the plurality of anchor members 130 may include two rectangular frames 1430 joining each other at a breadth to form a V-shape 1432, and the lengths of the two rectangular frames 1430 may form the end 132 that is sharp to penetrate the tissue. The rectangular frames 1430 and the V-shaped 1432 frame has a larger bearing area, and hence offers more resistance when the suture is being pulled. For example, when the suture is being pulled, the soft tissues inside the V-shaped frame 1432 gets “cramped” inside the V-shape 1432 and creates resistance against pulling. It is contemplated that the “gap” (empty space between the base plate 110 and the rectangular frame 1430 or V-shaped frame 1432) may provide an area for the tissue, e.g. tendon to heal through the gap, and the healed tissue, e.g. tendon may help to retain the implant.

FIGS. 15A to 15D show various view of another embodiment of the device 100. FIGS. 15A and 15B show perspective views of the device 100, FIG. 15C shows the top view of the base plate 110, and FIG. 15D shows a side view of the device 100. Device 100 may be based on device 10N described with reference to FIGS. 14A to 14D, and repeated descriptions will be omitted. For example, the base plate 110 and the central shaft 120 may be have the same configuration as that of device 100 described with reference to FIGS. 14A to 14D.

In the embodiment as shown in FIGS. 15A to 15D, each of the plurality of anchor members 130 may include an elongate portion 136 having a pointed tip 232 which may be sharp to form the end 132 that is configured to penetrate the tissue. For example, the elongate portion 136 may be shaped in the form of a needle which extends from the first surface 112 of the base plate 110, and the pointed tip 232 may allow a user, e.g. clinician to easily pierce the device 10O into the tissue for repair.

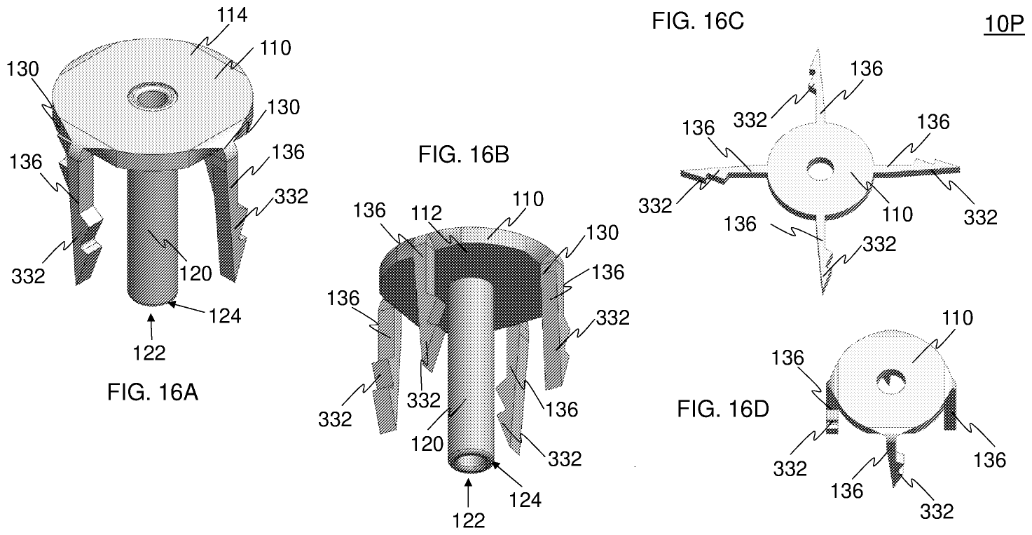

FIGS. 16A to 16D show various view of another embodiment of the device 10P. FIGS. 16A and 16B show perspective views of the device 10P, and FIGS. 16C and 16D show perspective views of the base plate 110 in an unfolded configuration and a folded configuration, respectively. Device 10P may share similarities with any of device 10A to 10O described with reference to FIGS. 1A to 15D, and repeated descriptions will be omitted.

Referring to FIGS. 16A to 16D, the base plate 110 may be a full circle, and the central shaft 120 may include a full circle extending from a portion, e.g. central portion, of the base plate 110. Each of the plurality of anchor members 130 may be arranged along the perimeter, for example, the circumference of the base plate 110. In some embodiments, each of the plurality of anchor members 130 may include an elongate portion 136 having a set of teeth 332 arranged along the length of the elongate portion 136. As mentioned above, the set of teeth 332 may form jagged and uneven edges which may act as a “hook or buckle” to provide a stronger anchorage of the device 10P into the tissue.

Referring to FIGS. 16C and 16D, each of the plurality of anchor members 130 may be configured to be foldable with respect to the base plate 110. For example, a user, e.g. clinician may fold each anchor member 130 towards the central shaft 120, for insertion into the tissue for repair. Accordingly, the clinician may individually adjust the angle of each of the plurality of anchor members 130 of the device 10P, and may provide a personalized device 10P. For example, the clinician may fold an anchor member 130 perpendicular to the base plate 110, and may fold another anchor member 130 at an angle to the base plate 110.

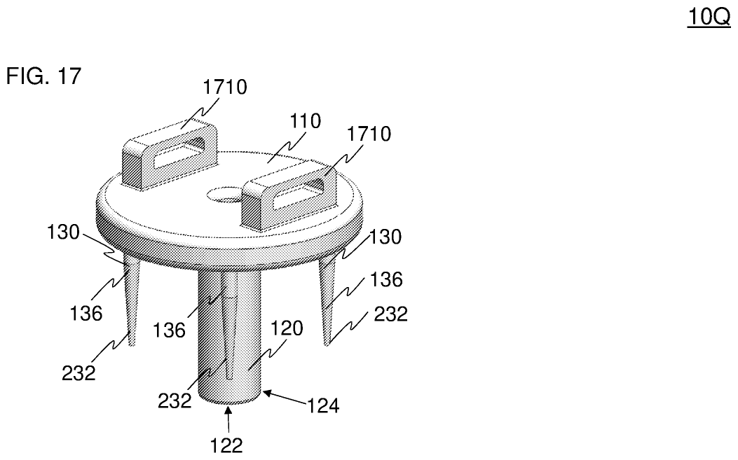

FIG. 17 shows the perspective view of another embodiment of the device 10Q. Device 10Q may be based on device 10P as described with reference to FIGS. 16A to 16D. For example, device 10Q may include a base plate 110 having a full circle, and a central shaft 120 extending from a portion, e.g. central shaft, of the base plate 110. The central shaft 120 may include a full circle configured to receive the suture.

Each of the plurality of anchor members 130 in device 10Q may include an elongate portion 136 having a pointed tip 232 which forms the end 132 of that is configured to penetrate the tissue. The elongate portion 136 may be shaped to form a needle having a pointed tip 232, which may allow the device 10Q to easily pierce and penetrate the tissue.

As shown in FIG. 17, the device 10Q may further include one or more mounting brackets 1710 extending from the second surface 114 of the base plate 110, and may be configured to extend away from the plurality of anchor members 130. The one or mounting brackets 1710 may be configured to be compatible to and to receive an applicator (as will be explained below), for delivering the device 10Q to the tissue for repair.

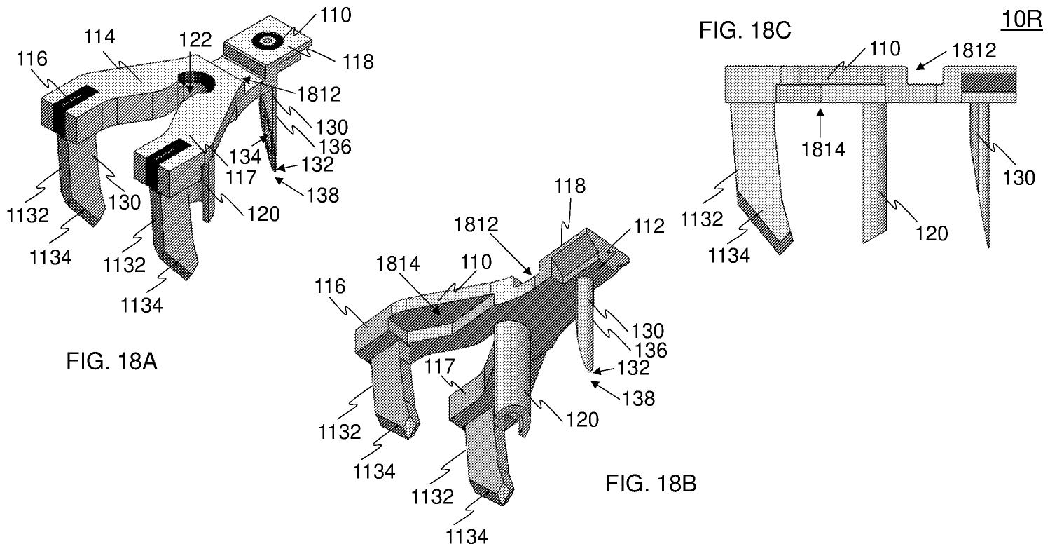

FIGS. 18A to 18C show various views of another embodiment of the device 10R. FIGS. 18A and 18B show the perspective views, and FIG. 18C shows the side view of the device 10R. Device 10R as shown in FIGS. 18A to 18C may be based on device 10A. For example, the base plate 110 and the central shaft 120 may be the same as device 10A, and the description of device 10R will thus focus on the variations of device 10R.

In device 10R, an anchor member 130 may differ to another anchor member 130. For example, an anchor member 130 may include an elongate portion 136, which may include a lumen 134, and may have a beveled tip 138. Another anchor member 130 may include the first portion 1132 extending from the first surface 112 of the first 116 and second 117 segments of the base plate 110, and a second portion 1134 extending from an end of the first portion 1132. The first portion 1132 may extend perpendicular to or at an angle to the base plate 110, and the second portion 1134 may extend towards a central axis, e.g. center of the base plate 110 or towards the central shaft 120. The first 1132 and second 1134 portions may have a slightly inward biased profile which is able to help prevent dislodging of device 10R from the tissue. In various embodiments, the curvature of the first 1132 and second 1134 portions may be configured to match the insertion path of the device 10R into the tissue site. Accordingly, the cutting area (on the tissue site) may be minimized, thereby reducing the force required, and amount of tissue that needs to be cut.

In the embodiment shown in FIGS. 18A to 18C, the third segment 118 may include a depression 1812, e.g. groove, on the second surface 114 of the base plate 110. The depression 1812 allows the applicator to easily hold the device 10R and prevents intended forward or backward motion of the device 10R while inserting the device 10R into tissue. Forming the depression 1812 on the third segment 118 may also reduce the overall manufacturing costs of the device 10R (e.g. when device is produced using injection molding), and at the same time, lowers the height of the device 10R. A shorter height may be helpful as there may be limited vertical space available at the injury site, e.g. shoulder, and the device 10R may be easily inserted into the tissue. In addition, at least one of the first 116 or second 117 segments may include a slot 1814 arranged on the first surface 112 of the base plate 110. The slot 1814 allows the applicator to hold the device 10R, and prevents the device 10R from dropping from the applicator during insertion.

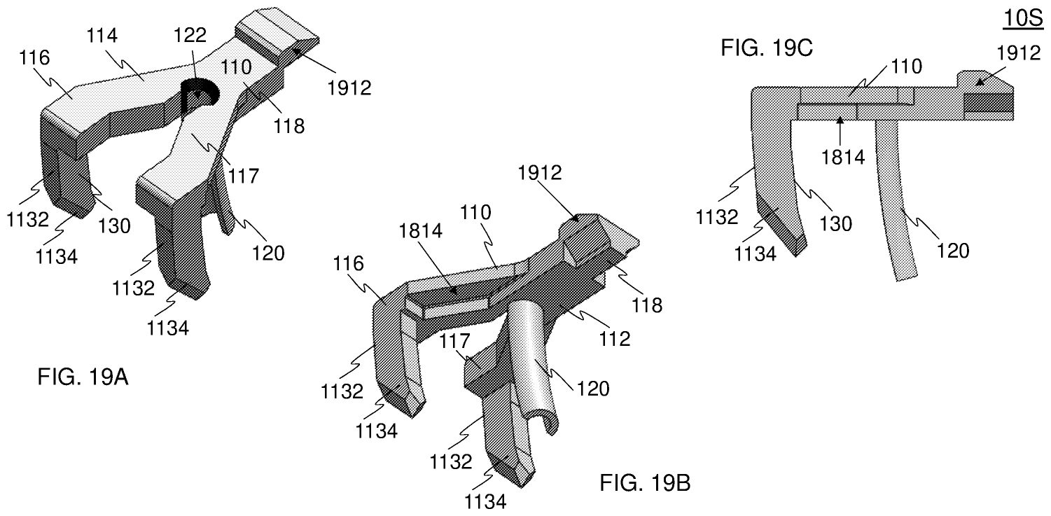

FIGS. 19A to 19C show various views of another embodiment of the device 10S. FIGS. 19A and 19B show the perspective views, and FIG. 19C shows the side view of the device 10S. Device 10S may be based on device 10R. For example, the base plate 110 including the slot 1814, and the anchor member 130 having the first portion 1132 and the second portion 1134 may be the same as device 10R. The description of device 10S will thus focus on the variations of device 10S.

In device 10S, the plurality of anchor members 130 may be arranged along a perimeter of the base plate 110. For example, the anchor members 130 may extend from the ends of each of the first 116 and second 117 segments. During manufacturing, the connection between the anchor member 130 and the first 116 and second 117 segments may be manufactured as a single body, i.e. one piece, instead of joint body, i.e. two separate pieces, which may be achieved using conventional machine processes. This reduces manufacturing costs and simplifies the manufacturing process. In device 10S, the central shaft 120 may be pivotable, e.g. arranged at an angle, with respect to a portion of the base plate 110. In some embodiments, the central shaft 120 may be curved. This may be helpful in transmitting the force evenly, from the suture to the central shaft 120, and onto the tissue, thereby minimizing bending of the central shaft 120 when the tissue is under tension. Further, the curved profile of the central shaft 120 reduces the angle at which the suture exits from the central shaft 120, and reduces the risk of cutting the suture during insertion and attachment of device 10S.

As shown in FIGS. 19A to 19C, the third segment 118 may further include a protruding step 1912 extending from an end of the second surface 114 of the third segment 118. The protruding step 1912 allows the applicator to hold the device 10S and prevents intended forward or backward motion when the device 10S is inserted into the tissue. The protruding step 1912 also increases the anchorage strength of the device 10S in the tissue, and prevents accidental dislodgement. The protruding step 1912 may further include a tapered profile, e.g. wedged-shaped profile, which prevents the device 10S from unintentionally cutting the suture during insertion into the tissue.

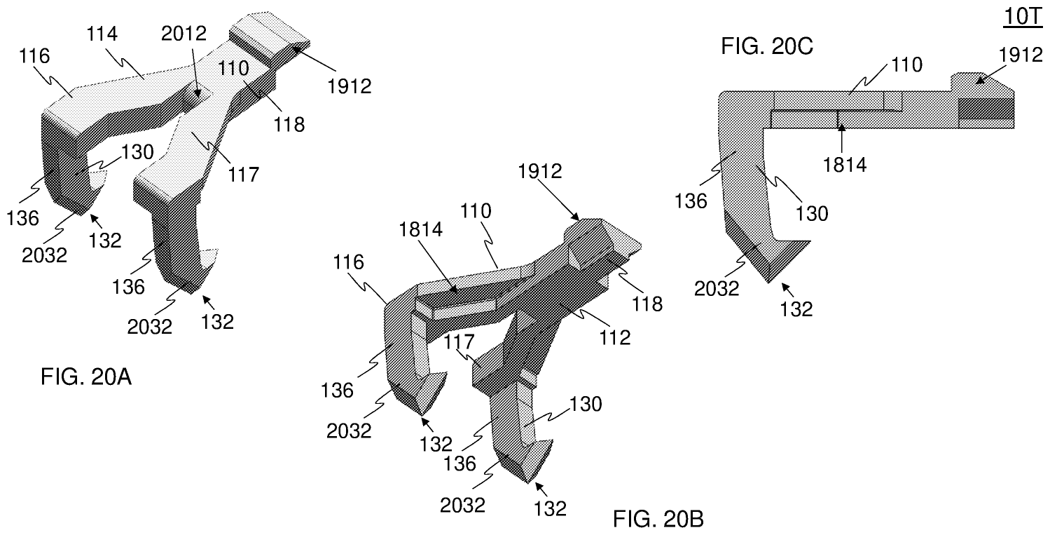

FIGS. 20A to 20C show various views of another embodiment of the device 10T. FIGS. 20A and 20B show the perspective views, and FIG. 20C shows the side view of the device 10T. Device 10T as shown in FIGS. 20A to 20C may be based on device 10S. For example, the base plate 110 including the slot 1814, and the protruding step 1912 of the third segment 118 may be the same as device 10S, and the description of device 10T will thus focus on the variations of device 10T.

In the embodiment as shown in device 10T, each of the plurality of anchor members 130 may include the elongate portion 136 extending from the first 116 and 117 second segments of the base plate 110, and may further include a first fluke 2032 (i.e. fluke is the part of the anchor member that may be attached to the tissue) arranged at the tip of the elongate portion 136 to form the end 132 configured to penetrate the tissue. The first fluke 2032 may form a “claw” mechanism that extends towards the central axis of the base plate 110. For example, the first fluke 2032 may be shaped to form an inward claw that acts as a “hook” to anchor the device 10T when embedded in tissue. Under tension, the first fluke 2032 may cooperate with the flat surfaces of the first 116 and second 117 segments to minimize the occurrence of accidental dislodgement of the device 10T. Specifically, the first fluke 2032 may be pulled inwards by a suture (during suturing of device 10T to tissue), to anchor the device 10T into tissue. As shown in FIGS. 20A to 20C, device 10T may not include the central shaft 120, as it is contemplated that the central shaft 120 may obstruct the inward pull of the first fluke 2032. Alternatively, it is contemplated that device 10T may include the central shaft 120.

Device 10T may also include a knob 2012 positioned at the interface where the first 116 and second 117 segments are connected. The knob 2012 may have a curved profile so as to prevent accidental cutting of the suture during device 10T insertion.

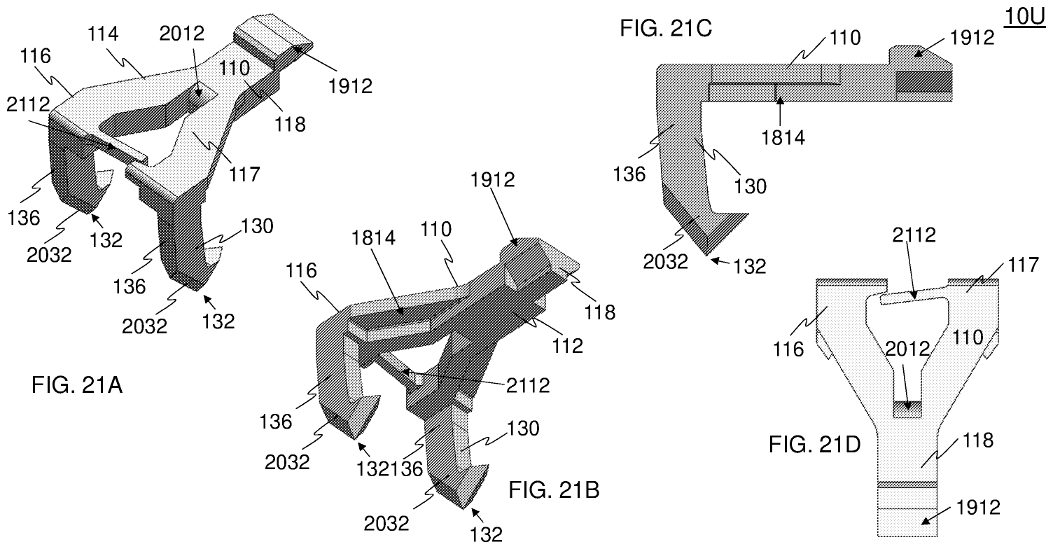

FIGS. 21A to 21D show various views of another embodiment of the device 10U. FIGS. 21A and 21B show the perspective views, FIG. 21C shows the side view of the device 10U, and FIG. 21D shows the top view of the base plate 110. Device 10U as shown in FIGS. 21A to 21D may be based on device 10T. For example, the base plate 110 including the slot 1814, the protruding step 1912 of the third segment 118, the anchor member 130 having the elongate portion 136 and the first fluke 2032, and the knob 2012 arranged at the interface of the first 116 and second 117 segments may be the same as device 10T. The description of device 10U will thus focus on the variations of device 10U.

Device 10U may further include a gate 2112 arranged to connect an end of the first segment 116 to an end of the second segment 117. For example, the gate 2112 may extend from an end of the first segment 116. The second segment 117 may include an extension, such that when connected, the gate 2112 abuts the extension of the second segment 117. In some embodiments, the gate 2112 may be further configured to move towards the interface where the first and second segments are connected. Thus, gate 2112 may be a one-way gate which moves towards the knob 2012 of the device 10U. The unidirectional gate 2112 may prevent the suture from detaching from the device 10U during insertion. In the event of accidental dislodgement, the gate 2112 may also prevent the device 10U from falling from the tissue, e.g. shoulder into another part of the body, e.g. arm. Alternatively, or in addition, it is contemplated that gate 2112 may move away from knob 2012 of device 10U, or may be bi-directional, e.g. two-way gate and may move away or towards the knob 2012 of the device 10U.

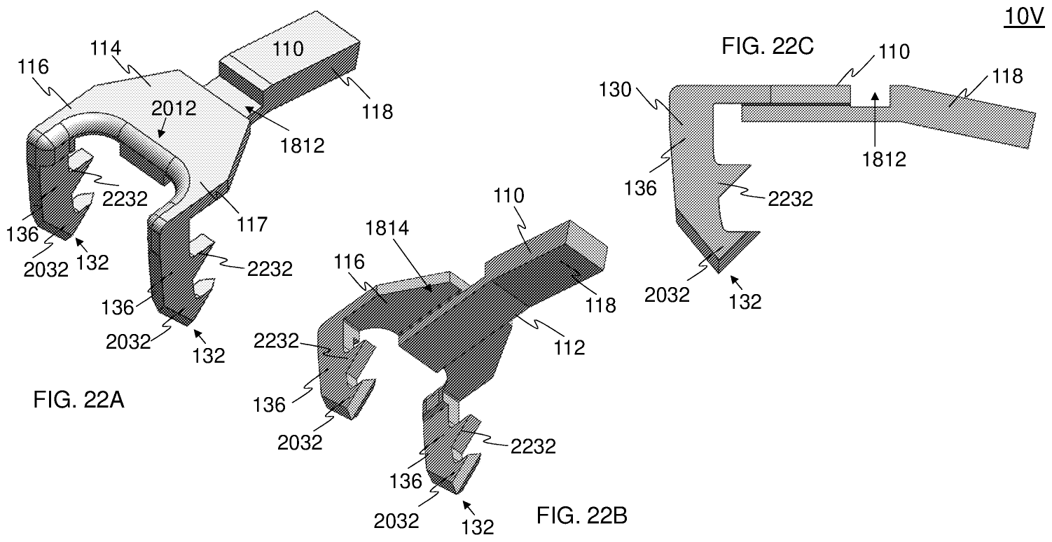

FIGS. 22A to 22C show various views of another embodiment of the device 10V. FIGS. 22A and 22B show the perspective views, and FIG. 22C shows the side view of the device 10V. Device 10V may share similarities to any of devices 10R to 10U. For example, the third segment 118 may include the depression 1812, and the anchor member 130 may include the elongate portion 136 and the first fluke 2032. The description of device 10V will thus focus on the variations of device 10V.

In device 10V, the knob 2012 may be positioned at the interface where the first 116 and second 117 segments are connected, such that the size of the aperture 122 may be maximized. This may be useful for sutures with larger widths, for example, the flat suture having a width ranging from 2 mm to 5 mm. Device 10V may further include a second fluke 2232 arranged along a length of the elongate portion 136. The additional fluke 2232 may provide additional anchorage to prevent accidental dislodgement of the device 10V under tension.

In the embodiment as shown in FIGS. 22A to 22C, the third segment 118 may be configured to be pivotable with respect to the first 116 and second 117 segments of the base plate 110. The third segment 118 may extend at an angle with respect to the base plate 110, and may act as a pivot point to minimize the risk of accidental dislodgement of the device 10V. For example, the third segment 118 may further sink into the elastic tissue, e.g. tendon, (as opposed to dislodging from tissue) under tension, e.g. pulling of the suture. It is contemplated that the third segment 118 may be configured to move along a longitudinal axis of the device 10V.

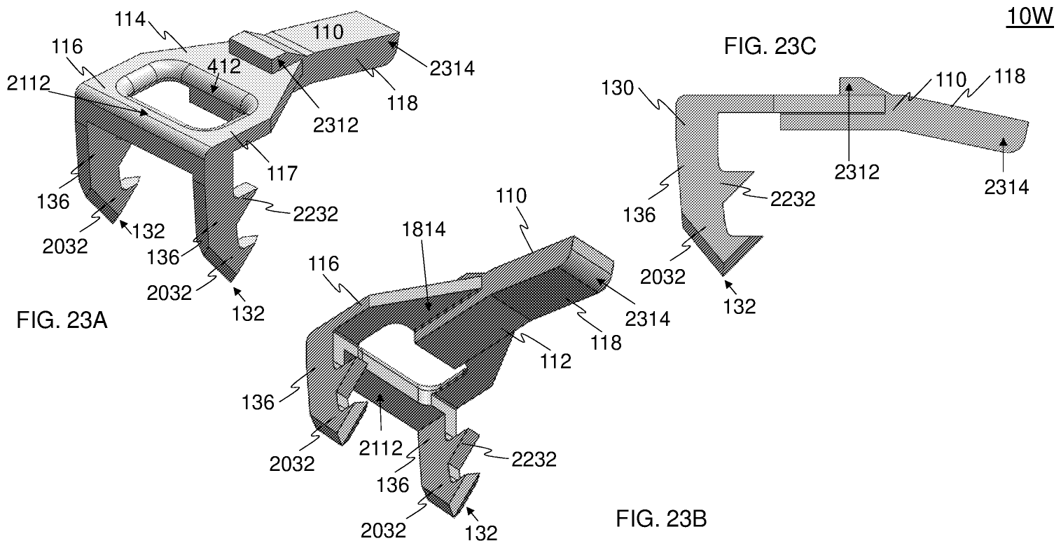

FIGS. 23A to 23C show various views of another embodiment of the device 10W. FIGS. 23A and 23B show the perspective views, and FIG. 23C shows the side view of the device 10W. Device 10W may be based on device 10V. For example, the third segment 118 may be pivotable with respect to the first 116 and second 117 segments, and the anchor member 130 may include the elongate portion 136, the first fluke 2032 and the second fluke 2232, and the description of device 10W will thus focus on the variations of device 10W.

In device 10W, the gate 2112 may be arranged to connect the end of the first segment 116 to the end of the second segment 117. In this embodiment, the gate 2112 may be a permanent barrier and may not be moveable. In addition, the third segment 118 may include an additional step protrusion 2312 arranged along a length of the third segment 118. The step protrusion 2312 may provide additional stiffness and further anchorage when the device 10W is embedded in tissue. In device 10W, an end 2314 of the third segment 118 may be curved so as to minimize the risk that a part of the third segment 118 cuts into the tissue upon application of a force, e.g. by the applicator, for example during device 10W insertion.

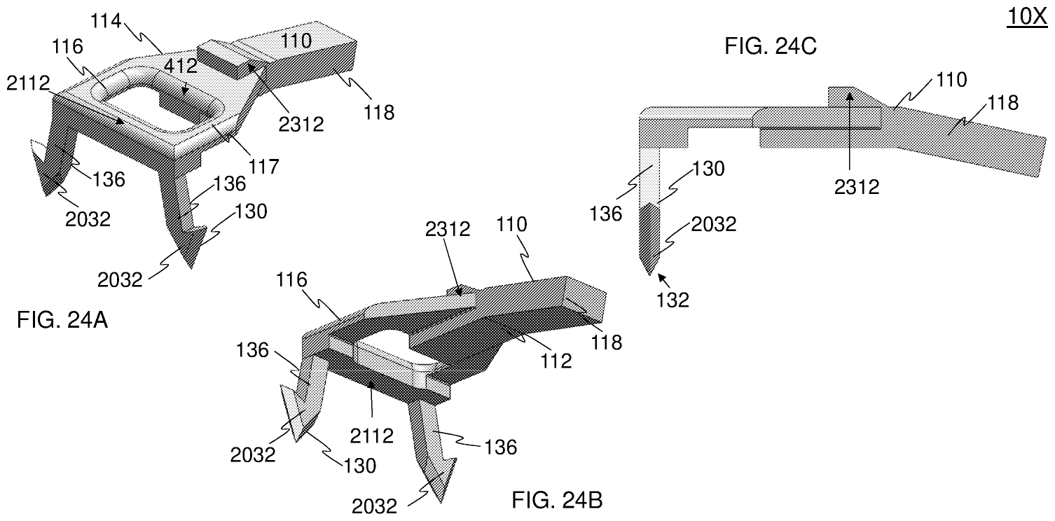

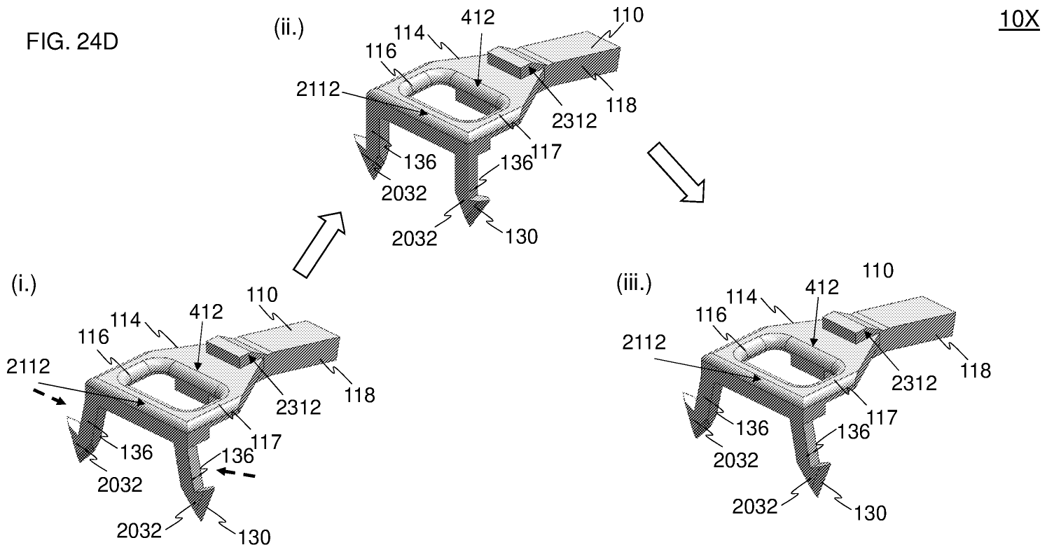

FIGS. 24A to 24D show various views of another embodiment of the device 10X. FIGS. 24A and 24B show the perspective views, and FIG. 24C shows the side view of the device 10X. FIG. 24D shows the device 10X as it is inserted into the tissue. Device 10X as shown in FIGS. 24A to 24D may be based on device 1OW, but may not include the second fluke 2232 and the curved end 2314 of the third segment 118. The description of device 10X will thus focus on the variations of device 10X.

In device 10X, the elongate portion 136 may extend at an angle to the first 116 and second 117 segments, and the first fluke 2032 may be extend away from the central axis of the base plate 110. Referring to FIG. 24D (i.), the elongate portions 136 of the anchor members 130 may be held between a pair of rods, e.g. of the applicator, which applies a lateral force (see dotted arrows) on the elongate portions 136. As shown in FIG. 24D (ii.), the anchor members 130 may move towards the central axis of the base plate 110 and may extend perpendicular to the base plate 110 as it is inserted into the tissue. Upon release of the applicator, the anchor members 130 may move into their original configuration, where the elongate portion 136 and the first fluke 2032 flexes away from the central axis of the base plate 110 (see FIG. 24 (iii.)). In this embodiment, the anchor members 130 act as a “hook” to anchor the device 10X into the tissue, and to minimize the risk of device 10X dislodgement.

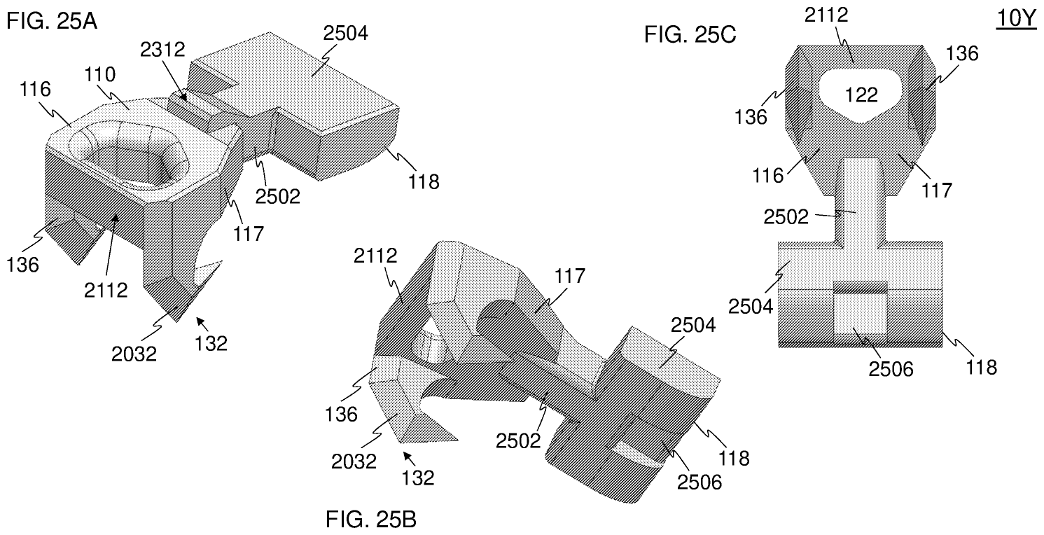

FIGS. 25A to 25C show various views of another embodiment of the device 10Y. FIGS. 25A and 25B show the perspective top and bottoms views, respectively, and FIG. 25C shows the bottom view of device 10Y. Device 10Y may be based on any of device 10T, 10U, or 10X, which includes a base plate 110 having the first 116, second 117 and third 118 segments, a first fluke 2032 arranged at the tip of the elongate portion 136 to form the end 132 configured to penetrate the tissue, a gate 2112 arranged to connect the end of the first segment 116 and the end of the second segment 117, and a step protrusion 2312 arranged along the length of the third segment 118. The description of device 10Y will thus focus on the variations of device 10Y.

In device 10Y, the third segment 118 of the base plate 110 may include a first bar 2502 extending from the portion of the base plate 110 having the aperture 122, and a second bar 2504 extending perpendicular to and from an end of the first bar 2502. The third segment 118 comprising the first bar 2502 and second bar 2504 may be integrally molded to form a “T-shape or wine-glass shape”, and may be pivotally attached to the first 116 and second 117 segments of the base plate 110. In various embodiments, the first bar 2502 and the second bar 2504 of device 10Y may have an elongated profile to facilitate device 10Y retention in the tissue for repair. In various embodiments, integrally molding the first bar 2502 and second bar 2504 may reduce manufacturing costs. Alternatively, the first bar 2502 and the second bar 25024 may be molded as separate pieces and arranged to form the “T-shape or wine-glass shape”. It is contemplated that the second bar 2504 may be pivotally attached to the first bar 2502 and in some embodiments, the first bar 2502 and the second bar 2504 may be arranged to form a Y-shape. It is further contemplated that the first bar 2502 and the second bar 2504 may not be pivotally attached to the first 116 and second 177 segments.

Device 10Y may further include a slot 2506 arranged on a first surface, e.g. surface which may be in contact with a tissue for repair, of the second bar 2504. The slot 2506 may be used to facilitate alignment of the device 10Y to an applicator (as will be explained below), and the fixing of said device 10Y to a tissue for repair.

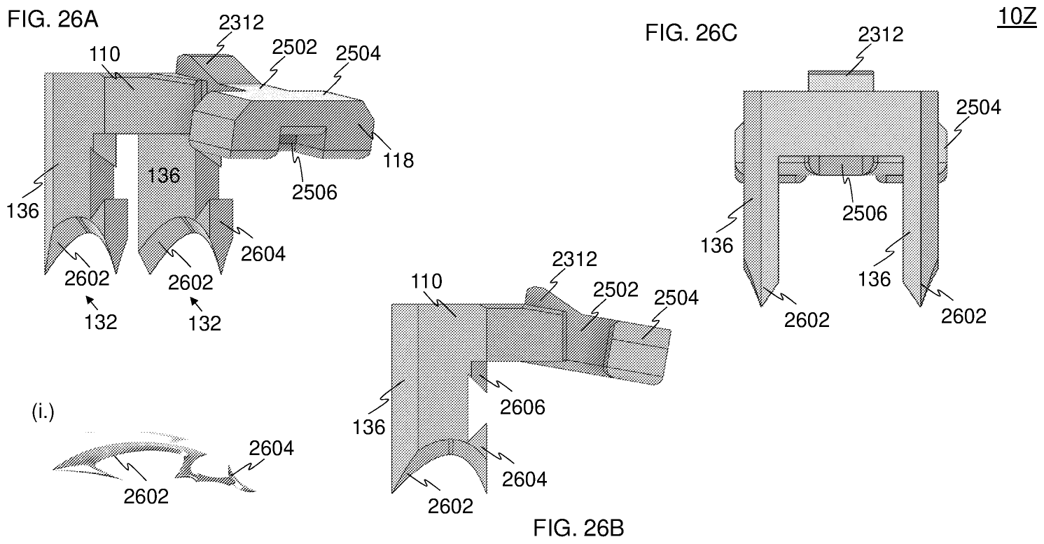

FIGS. 26A to 26C show various views of another embodiment of device 10Z. FIG. 26A shows a perspective view and inset (i.) shows an exploded view of a sickle-head tip 2602 and the third fluke 2604 of device 10Z. FIG. 26B shows a side view, and FIG. 26C shows a front view of device 10Z. Device 10Z may be based on device 10Y, and the description will focus on the variations of device 10Z.

In device 10Z, the plurality of anchor members 130 having the elongate portion 136 may include a sickle-head tip 2602 arranged at the end of the elongated portion 136, which forms the end 132 configured to penetrate tissue. The sickle-head tip 2602 may have a concave or crescent shape, e.g. a “hook-shaped” blade, which may aid in penetrating through tough tissue by reducing the surface area of the end 132 which penetrates the tissue. In particular, the sickle-head tip 2602 acts as a “hook or buckle” to secure device 10Z into the tissue, and strengthens the anchorage provided by said device 10Z.

In various embodiments, device 10Z may further include a third fluke 2604 arranged on an end of the sickle-head tip 2602, and a fourth fluke 2606 arranged along the length of the elongate portion 136. The fourth fluke 2606 may be arranged at a junction where the elongate portion 136 extends from the base plate 110. The third fluke 2604 and fourth fluke 2606 may form an upward “claw” mechanism that acts as a “hook” to anchor device 10Z when embedded in tissue, and may therefore prevent device 10Z from dislodging from the tissue.

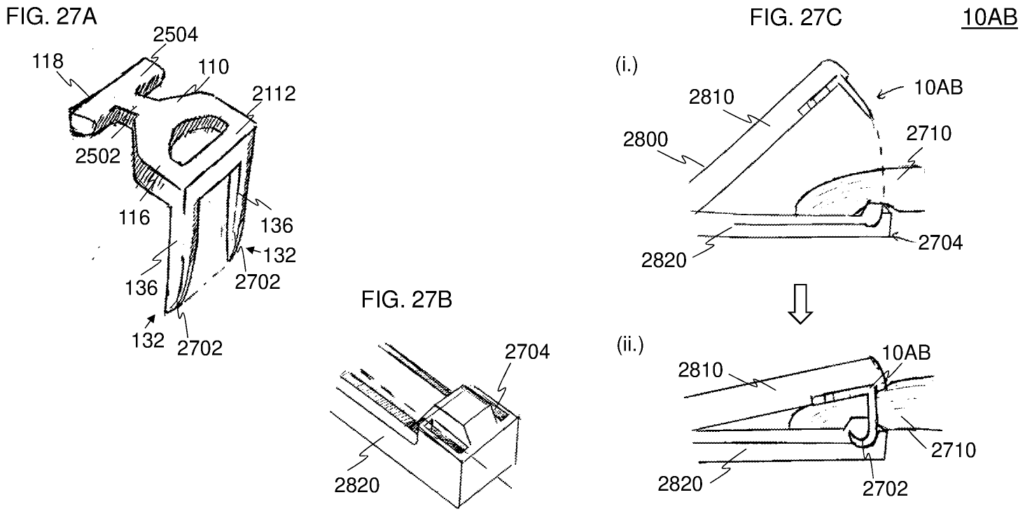

FIGS. 27A to 27C show various views of another embodiment of device 10AB. FIG. 27A shows a perspective view of device 10AB, FIG. 27B shows a platform 2820 of an applicator 2800 having an anvil 2822 configured to crimp the end 132 of the elongate portion 136 of device 10AB, and FIG. 27C shows side views of the device AB, (i.) before and (ii.) after entry into a tissue for repair. Device 10AB may be based on any of device 10Y or 10Z, and the description will focus on the variations of device 10AB.

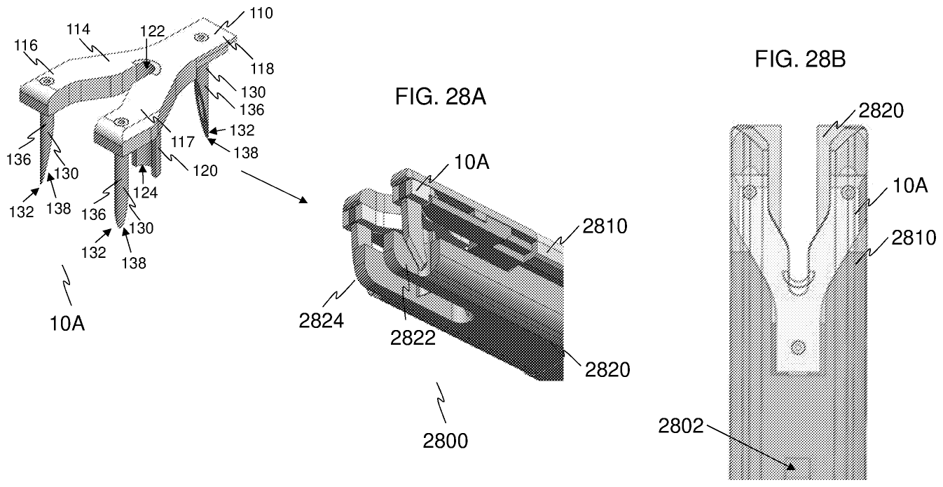

In device 10AB, an end 2702 of the elongate portion 136 may be configured to crimp or curve inwards, towards a central axis of the base plate 110, in response to a force applied against the end of the elongate portion 136. As will be elaborated below with reference to FIG. 28, delivery of the device 10AB may be facilitated by an applicator 2800 having a fastener 2810 and a platform 2820. Referring to FIG. 27B, the platform 2820 may include an anvil 2704 which is configured to crimp the end 2702 of the elongate portion 136, such that said end 2702 curves inwards and towards a central axis of the base plate 110. For example, with reference to FIG. 27C(i.), device 10AB may be attached to a fastener 2810, and a force may be applied to the fastener 2810 to drive device 10AB through a tissue 2710 resting on a platform 2820 of the applicator 2800. Accordingly, as shown in FIG. 27C(ii.), when the device 10AB penetrates through the tissue 2704 and is forced against the anvil 2704 on the platform 2820, said force causes the end 2704 of the elongate portion 136 to crimp towards the central axis of the base plate 110. The end 2704 of the elongate portion 136 may therefore act as a “hook or buckle” to anchor the device 10AB into the tissue 2704.

While various shapes, e.g. circular, Y-shaped, T-shaped (wine-glass shaped) third segment 118, of the base plate 110 have been described with reference to FIGS. 1A to 27C, it is contemplated that the base plate may be of various shapes, e.g. H-shaped. In various embodiments, the base plate 110 is configured to spread and optimize the distribution of forces to minimize or reduce the risk of disengagement of the device 10A to 10AB from the tissue.

The device 10A to 10AB of the various embodiments of the disclosure as described with reference to FIGS. 1A to 27C may be formed of a biocompatible material, for example a non-allergenic material which does not elicit detrimental local or systemic responses in a patient. In some embodiments, at least one part of the device 10A to 10AB may include or be formed of a biocompatible material. Non-limiting examples of biocompatible materials include: biocompatible metallic materials such as bio-absorbable metals, e.g. bio-absorbable magnesium; titanium; tantalum; surgical steel; nitinol; cobalt-chrome and metal alloys thereof; biocompatible polymers such as non bio-absorbable plastics, e.g. polycarbonate, polyether ether ketone; bio-absorbable plastics, e.g. polylactic acid, polyglycolide; ceramic biomaterials. The biocompatible material may be selected in accordance with the required strength and thickness for the device 10A to 10AB to penetrate and be securely anchored into the tissue. For example, the device 10A to 10AB may have a tensile strength in the range of 20 to 1200 MPa; and a Young Modulus's in the range from 1.5 to 250 GPa. In some embodiments, the selected biocompatible material may be malleable or may be flexible.

The device 10A to 10AB of the various embodiments of the disclosure as described with reference to FIGS. 1A to 27C may have a base plate 110 having a thickness ranging from 0.2 to 3 mm; a height of each anchor member 130 and the central shaft 120 may range from 0.2 to 30 mm.

FIGS. 1A to 27C show the various exemplary embodiments of the device 10A to 10AB for tissue repair. While the devices 10A to 10AB show the specific pairings the base plate 110, the central shaft 120, and each of the plurality of anchor members 130, the features that are described in the context of an embodiment may correspondingly be applicable to the same or similar features in the other embodiments, even if not explicitly described in these other embodiments. Furthermore, additions and/or combinations and/or alternatives as described for a feature in the context of an embodiment may correspondingly be applicable to the same or similar feature in the other embodiments.

According to another aspect of the disclosure, there is provided a kit of parts of a tissue repair assembly including a device for tissue repair, comprising a base plate having a first surface and a second surface opposing the first surface; an aperture arranged on a portion of the base plate, the aperture configured to receive a suture; a plurality of anchor members extending from the first surface of the base plate, each one of the plurality of anchor members having an end configured to penetrate the tissue, wherein each of the plurality of anchor members is spaced apart from another anchor member. The device for tissue repair may be the device 10A to 10AB described with reference to FIGS. 1A to 27C, and repeated descriptions will be omitted for brevity.

In various embodiments, the kit of parts may further include an applicator configured to receive and deliver the device for tissue repair to an injury site. The applicator may be configured to be compatible to the device, for example, the base plate of the device, the slot, groove or protrusion extending from the second surface of the base plate, and/or the one or more mounting brackets of the device. In some embodiments, the applicator may further include an effector configured to secure the device to the applicator.

In various embodiments, the kit of parts may further include an installation device for facilitating the attachment of the device to the applicator. The installation device may be configured to be compatible to the device, for example, the various base plate and/or anchor member embodiments as described with reference to device 10A to 10AB of FIGS. 1A to 27C.

Various non-limiting embodiments of the applicator, including in some embodiments, the effector and/or the installation module will be discussed with reference to FIGS. 28A to 33C.

FIGS. 28A and 28B show an example of a kit of parts of a tissue repair assembly 20A comprising the device 10A as described with reference to FIGS. 1A to 1C. FIG. 25A shows the side view, and FIG. 25B shows the top view applicator 2800 including the device 10A.

Referring to FIGS. 28A and 28B, the applicator 2800 may include a fastener 2810 dimensioned and sized to hold the base plate 110 of the device 10A. For example, the fastener 2810 may be shaped to receive the first 116, second 117 and third 118 segments of the base plate 110 of device 10A, wherein the first 116 and second 117 segments are spaced apart by the first angle 119a, and the second 117 and third 118 segments, and the first 116 and third 118 segments are each spaced apart by the second angle 119b.

The applicator 2800 may also include a platform 2820 arranged opposite the fastener 2810. In some embodiments, the platform 2820 may further include a suture passer or suture passer needle, e.g. needle arranged on or part of the suture passer (not shown), for threading the suture through the device 10A. The platform 2820 may have a positioning area 2824 having a plurality of holes 2822 for receiving the plurality of anchor members 130 of the device 10A. For example, the plurality of holes 2822 may be sized and dimensioned accordingly to receive the elongate portion 136 and the beveled tip 138 which forms the end 132 configured to penetrate the tissue. The positioning area 2824 including the plurality of holes may assist in the positioning and alignment of the device 10A for tissue repair.

In some embodiments, the fastener 2810 is positioned along a length of the platform 2820 via a first pivotable interface 2802. For example, the fastener 2810 may be attached to the platform 2820 by a hinge or a pin such that it may rotate towards or away from the fastener 2810. Accordingly, the applicator 2800 may be configured such that an application of a force on the fastener 2810 may cause the fastener 2810 to move towards the positioning area 2824, to secure the device 10A into the tissue. In some embodiments, the fastener 2810 may be configured to move away from the positioning area upon release of the force, leaving the device 10A secure to the tissue for repair.

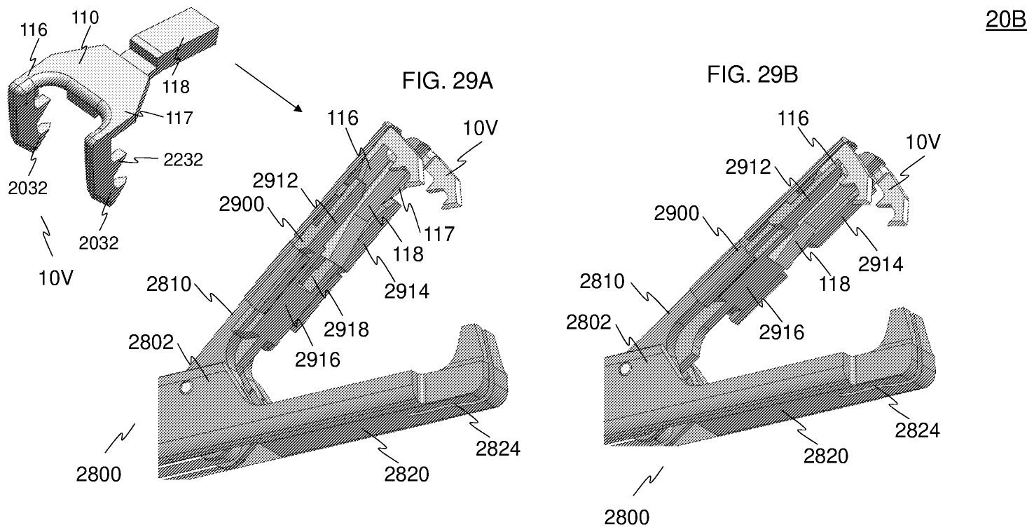

FIGS. 29A and 29B show an example of a kit of parts of a tissue repair assembly 20B comprising device 10V as described with reference to FIGS. 22A to 22C, and an applicator 2800 including an embodiment of an effector 2900. FIG. 29A shows the effector 2900 in an unlocked configuration, and FIG. 29B shows the effector 2910 in a locked configuration. The applicator 2800 of tissue repair assembly 20B may be based on the applicator 2800 as described with reference to FIGS. 28A and 28B.

In some embodiments, the fastener 2810 may further include an effector 2900 arranged along a length of the fastener 2810, and the effector 2900 may be dimensioned and sized to hold the base plate 110 of device 10V. As shown in FIG. 29A, the effector 2910 may include a first beam 2912, a second beam 2914, and a third beam 2916, the first beam 2912 and the second beam 2914 extending from a part of the third beam 2916, such that the first 2912, second 2914 and third 2916 beams form an opening 2918, e.g. a “U-shaped” opening, configured to receive the third segment 118 of the base plate 110 of device 10V. In the unlocked configuration, effector 2900 may be proximal to the first pivotable interface 2802, to allow a user to attach device 10V to an end of the fastener 2810.

Referring to FIG. 29B, a force applied on the fastener 2810 to move the fastener 2810 towards the positioning area 2824 of the platform 2820, e.g. downward force with respect to the fastener 2810, may cause the effector 2900 to move into a locked configuration to secure device 10V to the applicator 2800. For example, the force may cause the effector 2910 to slide along the fastener 2810 or part thereof, to move towards the end of the fastener 2810 holding the device 10V, such that the first beam 2912 and the second beam 2914 each hold the first segment 116, and the second segment 117 of the base plate 110, respectively, and the third segment 118 fits within the opening 2918 of the effector 2900. Accordingly, the device 10V may be secured to the fastener 2810 via effector 2900, and may be delivered to an injury site for tissue repair.

While effector 2900 has been described with reference to device 10V, effector 2900 may also be particularly applicable for device 10R, 10S, 10T, 10U, 10W and 10X, described with reference to FIGS. 18A to 21D, and FIGS. 23A to 24D.

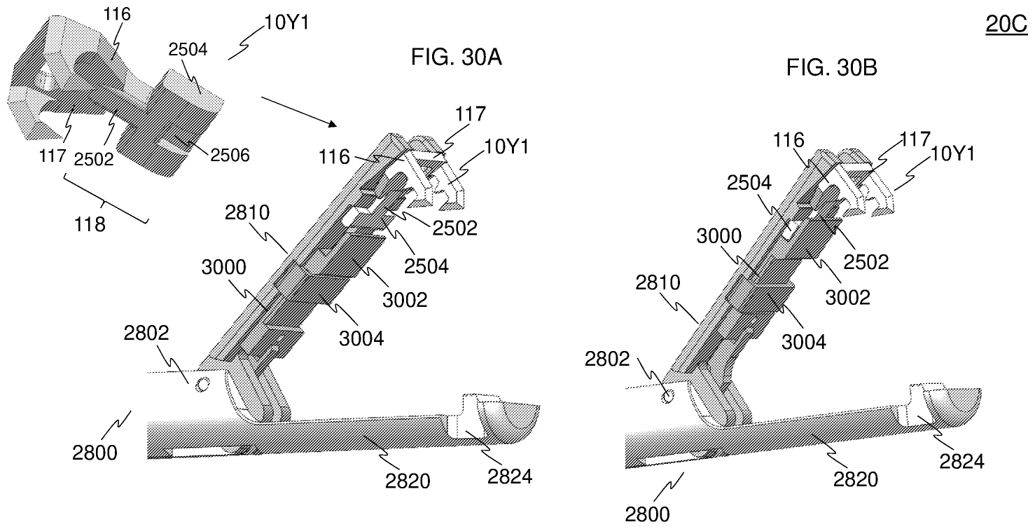

FIGS. 30A and 30B show an example of a kit of parts of a tissue repair assembly 20C comprising the base plate 110 of device 10Y as described with reference to FIGS. 25A to 25C, and an applicator 2800 including an embodiment of an effector 3000. In this embodiment, device 10Y1 may be the similar to device 10Y, and may further include a second fluke 2232 arranged on the length of the elongate portion 136 of the plurality of anchor members 130. FIG. 30A shows the effector 3000 in an unlocked configuration, and FIG. 30B shows the effector 3000 in a locked configuration. The applicator 2800 of tissue repair assembly 20C may be based on the applicator 2800 as described with reference to FIGS. 28A and 28B.

Referring to FIG. 30A, an effector 3000 may be arranged along a length of the fastener 2810, and may be dimensioned and sized to hold the base plate 110, in particular, the first bar 2502, and the second bar 2504 of the third segment 118 of device 10Y1. Effector 3000 may include a first plate 3002 configured to hold the third segment 118 of the base plate 110, and may include a first clasp 3004 arranged on the first plate 3002 and configured to slide along a length of the fastener 2810 or part thereof. In the unlocked configuration, the first plate 3002 may be proximal to the first pivotable interface 2802 and the first clasp 3004 may be in contact with the first plate 3002. A user may therefore attach device 10Y1 to an end of the fastener 2810.

Referring to FIG. 30B, a force applied on the fastener 2810 to move the fastener 2810 towards the positioning area 2824 of the platform 2820, e.g. downward force with respect to the fastener 2810, may cause the effector 3000 to move into a locked configuration to secure device 10Y1 to the applicator 2800. For example, the force may cause the first clasp 3004 to slide and move towards to the first pivotable interface 2802, and to cause the first plate 3002 to slide and move towards the end of the fastener 2810 holding the device 10Y1. As such, the first plate 3002 may encase and holds the third segment 118 of the device 10Y1. Accordingly, the device 10Y1 may be secured to the fastener 2810 via effector 3000, and may be delivered to an injury site for tissue repair.

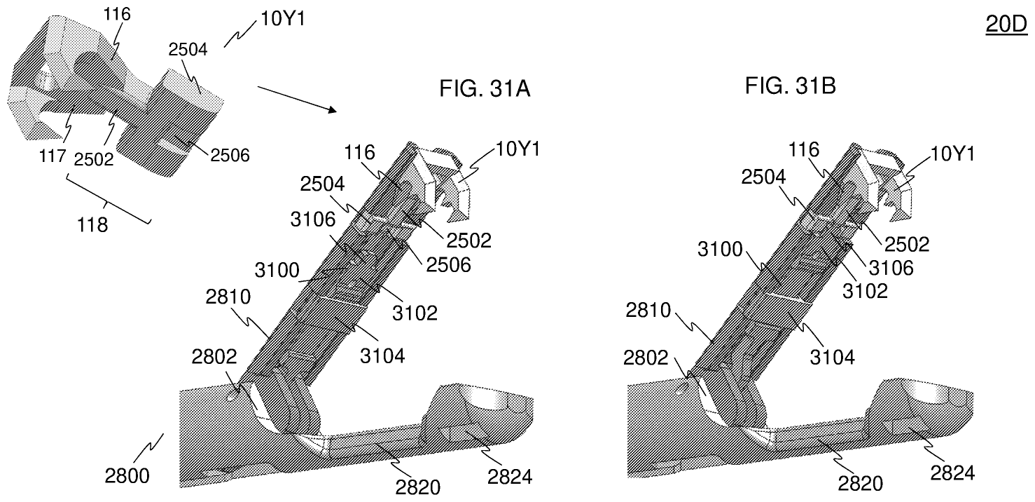

FIGS. 31A and 31B show an example of a kit of parts of a tissue repair assembly 20D comprising the base plate 110 of device 10Y as described with reference to FIGS. 25A to 25C, and an applicator 2800 including an embodiment of an effector 3100. Device 10Y1 may be based on device 10Y1 described with reference to FIGS. 30A and 30B. FIG. 31A shows the effector 3100 in an unlocked configuration, and FIG. 31B shows the effector 3000 in a locked configuration. The applicator 2800 of tissue repair assembly 20C may be based on the applicator 2800 as described with reference to FIGS. 28A and 28B.

Referring to FIG. 31A, an effector 3100 may be arranged along a length of the fastener 2810, and may be dimensioned and sized to hold the base plate 110, in particular, the slot 2506 arranged on the second bar 2504 of the third segment 118 of device 10Y1. Effector 3100 may include a second plate 3102 having a protrusion 3106 dimensioned to fit the slot 2506 of the third segment 118 of the base plate 110, and may further include a second clasp 3104 arranged on the second plate 3102 and configured to slide along a length of the fastener 2810 or part thereof. In the unlocked configuration, the second plate 3102 may be proximal to the first pivotable interface 2802, and the second clasp 3104 may be in contact with a part of the second plate 3102. Accordingly, a user may attach device 10Y1 to an end of the fastener 2810.

Referring to FIG. 31B, a force applied on the fastener 2810 to move the fastener 2810 towards the positioning area 2824 of the platform 2820, e.g. downward force with respect to the fastener 2810, may cause the effector 3100 to move into a locked configuration to secure device 10Y1 to the applicator 2800. For example, the force may cause the second clasp 3104 to slide and move towards to the first pivotable interface 2802, and to cause the second plate 3102 to slide and move towards the end of the fastener 2810 holding the device 10Y1, such that the protrusion 3106 arranged on the second plate 3102 fits into the slot 2506 of the third segment 118 of device 10Y1. Accordingly, device 10Y1 may be secured to the fastener 2810 via effector 3100, and may be delivered to an injury site for tissue repair.

While effector 3000 and 3100 (FIGS. 30A to 31B) have been described with reference to device 10Y1, effector 3000 and 3100 may be particularly applicable for device 10Y, 10Z and 10AB, described with reference to FIGS. 25A to 27C, wherein the third segment 118 has a T-shaped (wine-glass shaped) profile.

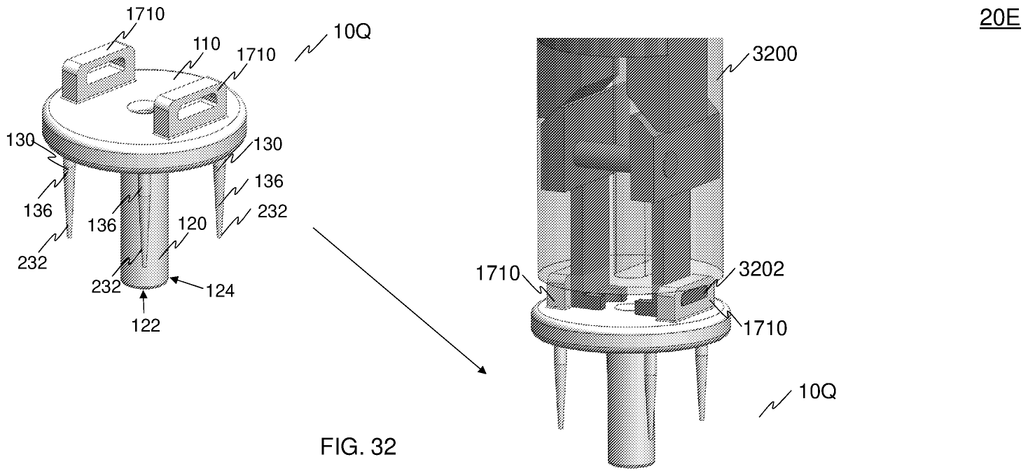

FIG. 32 shows another embodiment of an exemplary kit of parts of a tissue repair assembly 20E comprising the device 10Q as described with reference to FIG. 17. Referring to FIG. 32, the applicator 3200 may include one or more mounting protrusions 3202 configured to receive the one or more mounting brackets 1710 extending from the second surface 114 of the base plate 110. For example, the one or more mounting protrusions 3202 may fit within an opening provided by the mounting bracket 1710 of the device 10Q to hold the device 10Q for delivery to the tissue for repair. In some embodiments, the one or more mounting protrusions 3202 may be further configured to release, e.g. via a mechanism contained within the applicator 3200, the one or more mounting brackets 1710 of the device 10Q, leaving the device 10Q secured to the tissue for repair.

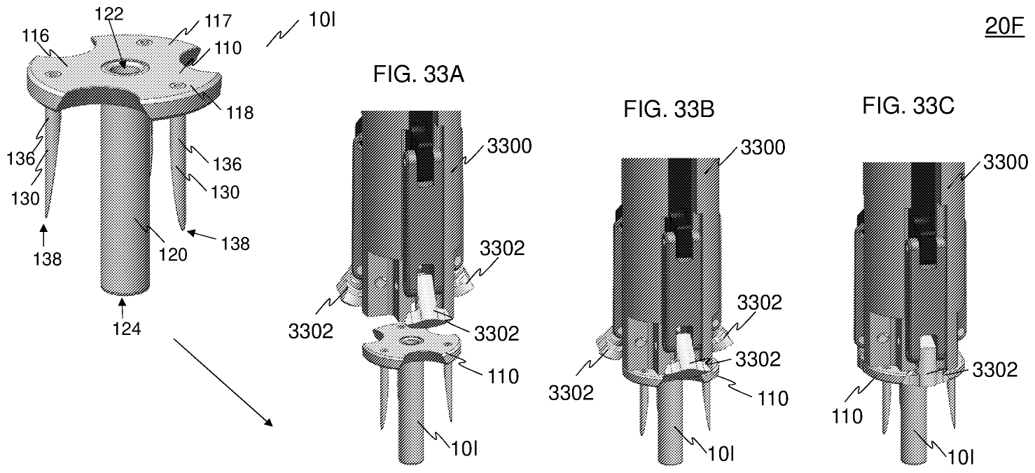

FIGS. 33A to 33C show another embodiment of an exemplary kit of parts of a tissue repair assembly 20F comprising the device 10I as described with reference to FIGS. 9A to 9D. Referring to FIGS. 33A to 33C, the applicator 3300 may include one or more mounting frames 3302 arranged at an end of the applicator 3300. The one or more mounting frames 3302 may be configured to hold the base plate 110 of device 10I, for delivery to the tissue for repair. For example, the one or more mounting frames 3302 may be shaped to hold the contours of the base plate 110, specifically, the arcs adjoining each of the first 116, second 117 and third 118 segments to each other. In some embodiments, the one or more mounting frames 3302 may be further configured to release, e.g. via a mechanism contained within the applicator 3300, the base plate 110, leaving the device 10I secured to the tissue for repair.

While non-limiting embodiments of applicators 2800 including in some embodiments, various designs of effectors 2900, 3000 and 3100, applicator 3200, 3300 have been described in FIGS. 28A to 33C, it is contemplated that applicators of various designs, including effectors thereof, may be configured for each of the devices 10A to 10AB described with reference to FIGS. 1A to 27C.

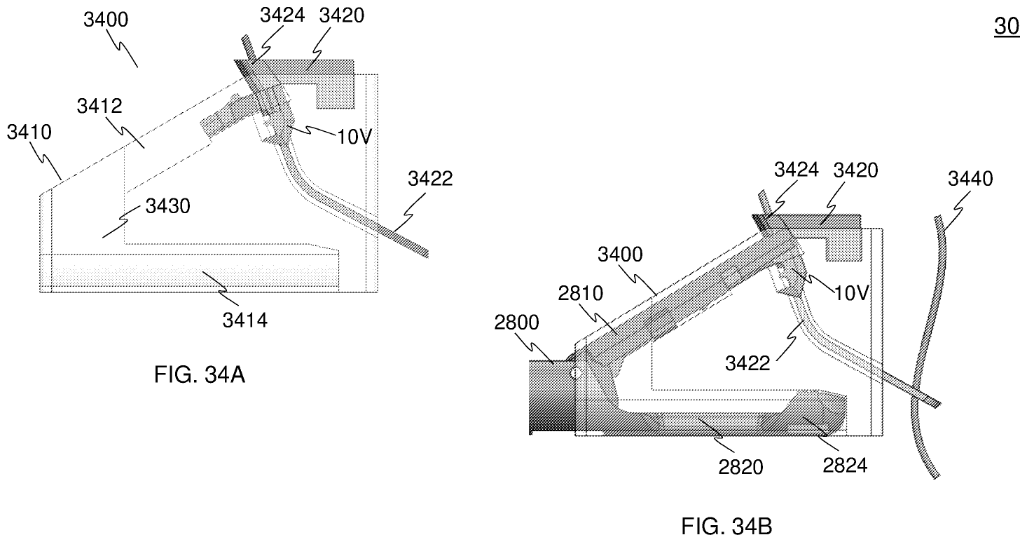

FIGS. 34A and 34B show another embodiment of an exemplary kit of parts of a tissue repair assembly 30 comprising an installation device 3400 and an applicator 2800. FIGS. 34A and 34B is described with reference to exemplary device 10V of FIGS. 22A to 22C, but embodiments are not limited thereto. FIG. 34A shows the installation device 3400, and FIG. 34B shows the installation device 3400 fitting within applicator 2800, for attaching device 10V to the fastener 2810 of the applicator 2800.

Referring to FIGS. 34A and 34B, the installation device 3400 may include a first module 3410 and a second module 3420. The first module 3410 may be dimensioned to fit between the fastener 2810 and the platform 2820 of the applicator 2800. In various embodiments, the first module 3410 may include a first component 3412, and a second component 3414 arranged opposite the first component 3412. In accordance with the design of applicator 2800, the first component 3412 and the second component 3414 may be connected to each other via a second pivotable interface 3430, and the second pivotable interface 3430 of the installation device 3400 may be configured to fit within the first pivotable interface 2802 of the applicator 2800.

The first component 3412 of the first module 3410 may be configured to receive the base plate 110 of device 10V for attaching said device 10V to the fastener 2810. In various embodiments, the installation module 3400 is configured such that an application of a force on the fastener 2810 to move the fastener 2810 towards the positioning area 2824 of the platform 2820, moves the effector 2900 (configured for device 10V) towards the end of the fastener 2810 to hold at least one of the first 116, second 117 or third 118 segments of the base plate 110 of the device 10V. As shown in FIGS. 29A to 29C, the effector 2900 may hold the first 116, and the second 117 segments, and the third segment 118 may fit in the opening 2918 of the effector 2900. Accordingly, the installation module 3400 may facilitate the attachment of the device 10V onto the fastener 2810.

In installation device 3400, the second module 3420 may be configured to be mobile and removable from the first module 3410. The second module 3420 may include a wire loop 3422 connected to a part of the second module 3420, and a hole 3424 arranged on the second module 3420. The wire loop 3422 may be configured to receive a suture 3440, and the hole 3424 may be configured to receive the wire loop 3422, and/or the wire loop 3422 having the suture 3440 threaded therethrough. In various embodiments, the hole 3424 of the second module 3420 may be positioned to be in alignment with the aperture 122 (configured to receive the suture 3440) of the base plate 110 of the device 10V. Accordingly, when the second module 3420 is removed from the first component 3412 holding the base plate 110 of the device 10V, the second module 3420 carries the wire loop 3424 and the suture 3440, through the hole 3424 of the second module 3420 and the aperture 122 of the base plate 110 of device 10V. As such, the suture 3440 may be threaded through device 10V.

While an embodiment of the installation device 3400, and the applicator 2800 including the effector 2900 configured for device 10V has been described with reference to device 10V of FIGS. 22A to 22C, embodiments of the disclosure are not limited thereto. In some other embodiments, the installation device may be designed to attach any of device 10A to 10AB described with reference to FIGS. 1A to 27C, to an appropriate applicator, for attachment of said device 10A to 10AB to the applicator.

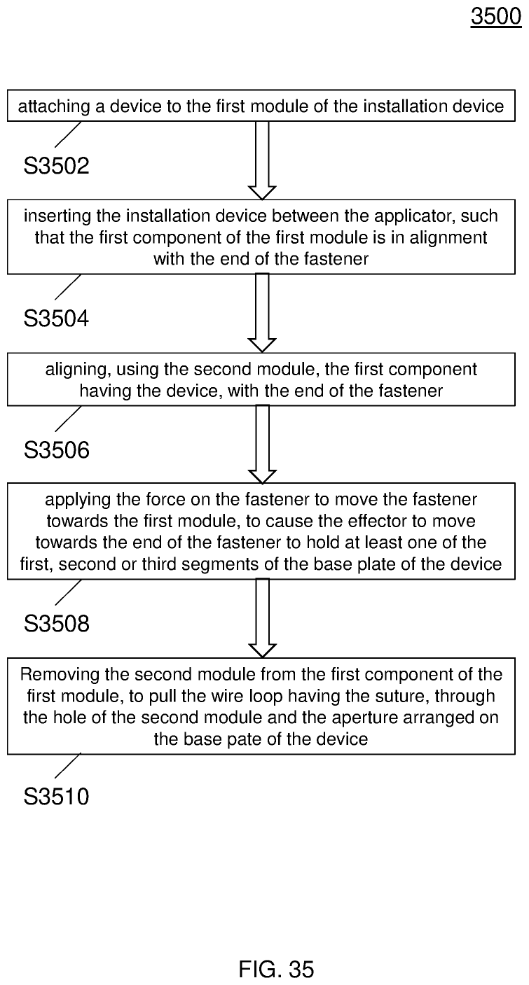

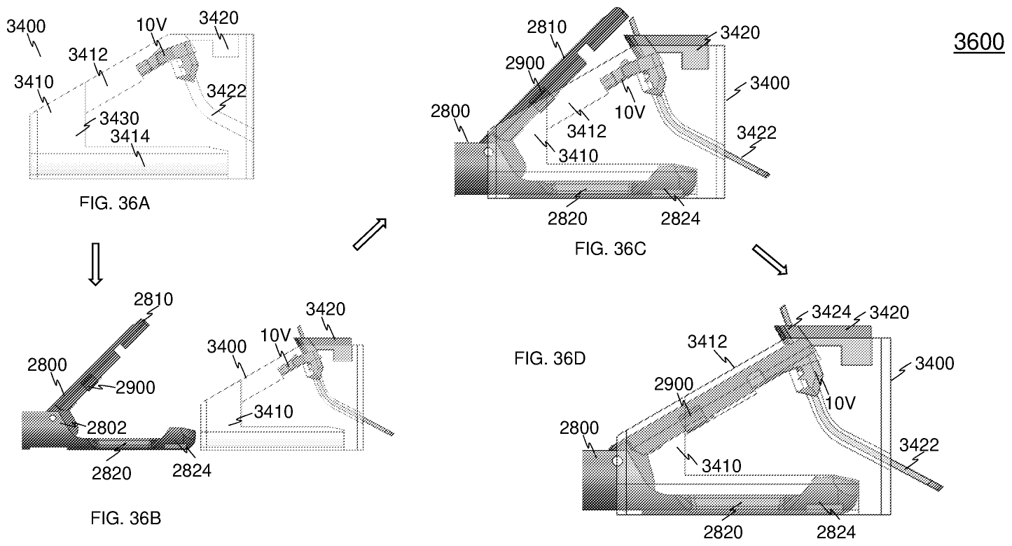

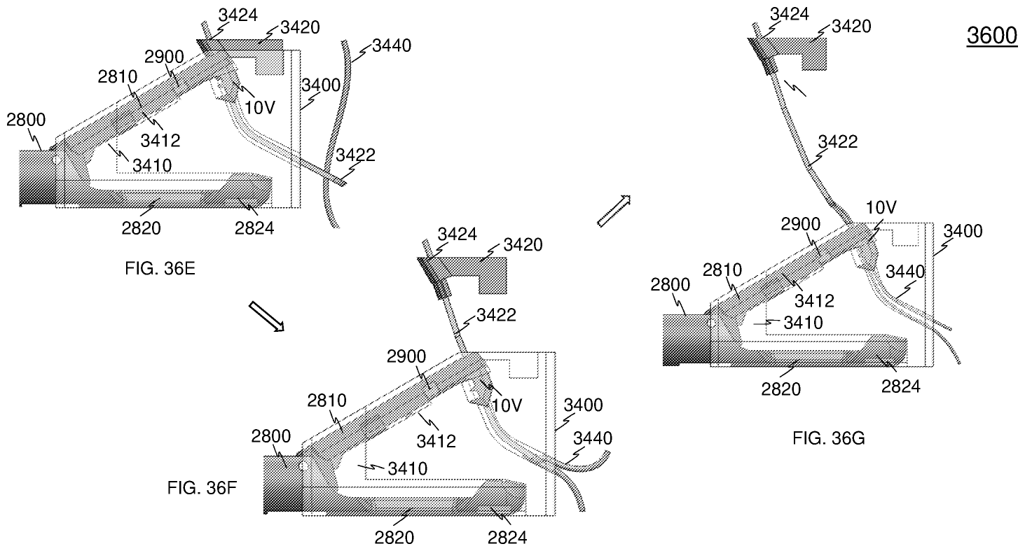

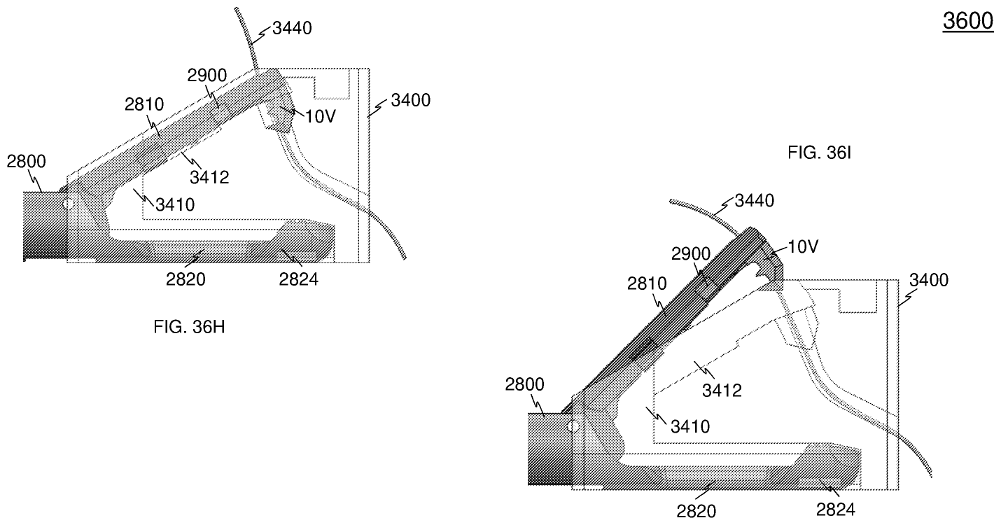

FIG. 35 shows an exemplary flowchart of a method 3500 of attaching a tissue repair device to an applicator. Method 3500 may be particularly applicable for the kit of parts of a tissue repair assembly including the tissue repair device, applicator and installation device. The device may correspond to any of device 10A to 10AB of FIGS. 1A to 27C, the applicator and effector thereof, and the installation device may be configured for said device 10A to 10AB. FIGS. 36A to 361 show exemplary schematics 3600 of method 3500 for attaching an exemplary tissue repair device, device 10V to the applicator 2800, and the removal of the installation device 3400 from the applicator 2800.

Referring to FIGS. 35 and 36A to 36E, method 3500 includes: (i.) attaching the tissue repair device to the first module of the installation device (step 3502, FIG. 36A); (ii.) inserting the installation device between the applicator, such that the first component of the first module is in alignment with the end of the fastener (step 3504, FIGS. 36B and 36C); (iii.) aligning, using the second module, the first component having the device, with the end of the fastener (step 3506, FIG. 36D); and (iv.) applying the force on the fastener to move the fastener towards the first module, i.e. positioning area on platform of applicator, to cause the effector to move towards the end of the fastener to hold at least one of the first, second or third segments of the base plate of the device (step 3508, FIG. 36E).

In various embodiments and with reference to step 3506 and FIG. 36D, aligning, using the second module, the first component having the tissue repair device to the end of the fastener may include, aligning the hole arranged on the second module to the aperture arranged on the base plate of the device.

In method 3500 and with reference to FIG. 36E, a suture may be threaded through the wire loop of the second module, and method 3500 may further include: (v.) removing the second module from the first component of the first module, to pull the wire loop having the suture, through the hole of the second module and the aperture arranged on the base plate of the device (step 3510, FIGS. 36F and 36G).

Referring to FIGS. 36H and 361, upon attachment of the tissue repair device to the applicator, method 3500 may further include removing the installation device from the applicator. In various embodiments, releasing the force applied on the fastener may lift the tissue repair device and the suture, out of the installation device. Accordingly, the tissue repair device and suture may be attached to the fastener of the applicator, for delivery to an injury site.

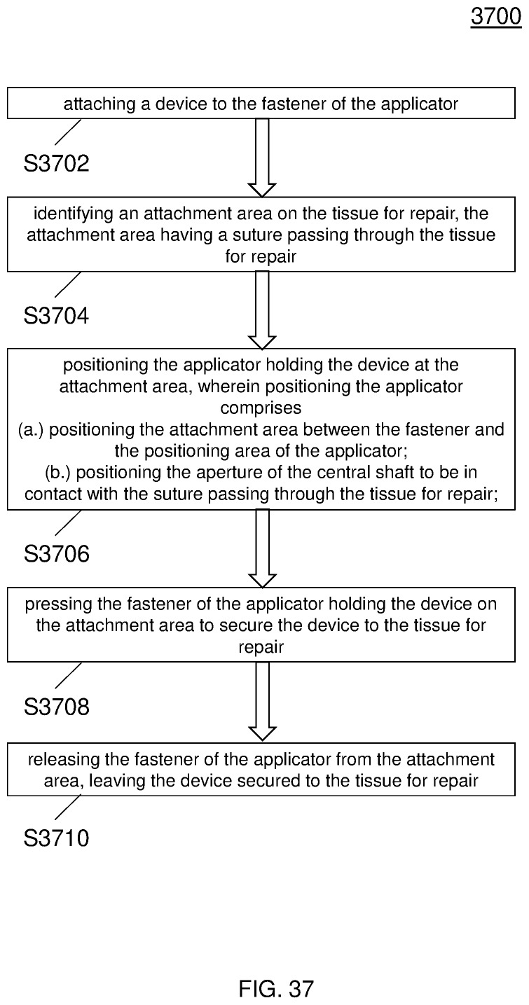

FIG. 37 shows an embodiment of a method 3700 of repairing a tissue, according to another aspect of the disclosure. The steps of method 3700 may be based on the device 10A described with reference to FIGS. 1A to 1C, and kit of parts including device 10A and the applicator 2800 as described with reference to FIGS. 28A and 28B. Method 3700 includes: (i.) attaching the device to the fastener of the applicator (step 3702); (ii.) identifying an attachment area on the tissue for repair, the attachment area having a suture passing through the tissue for repair (step 3704); (iii.) positioning the applicator holding the device at the attachment area, wherein positioning the applicator comprises (a.) positioning the attachment area between the fastener and the positioning area of the applicator, and (b.) positioning the aperture of the central shaft to be in contact with the suture passing through the tissue for repair (step 3706); (iv.) applying the force on the fastener of the applicator holding the device on the attachment area to secure the device to the tissue for repair (step 3708); and (v.) releasing the fastener of the applicator from the attachment area, leaving the device secured to the tissue for repair (step 3710). Method 3700 may be particularly applicable for devices including a central shaft 120, e.g. device 10A to 10S as described with reference to FIGS. 1A to 19C.

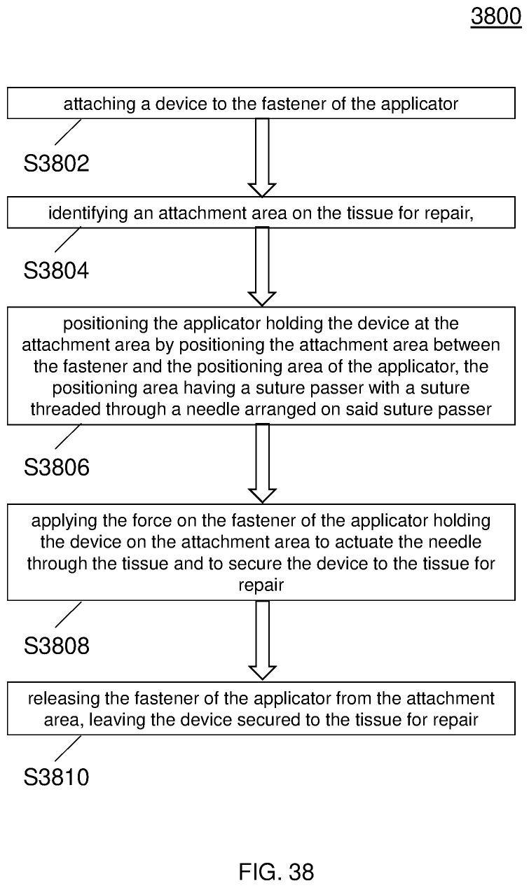

FIG. 38 shows another embodiment of method 3800 of repairing a tissue, according to another aspect of the disclosure. The steps of method 3800 may be based on any one of device 10R to 10W as described with reference to FIGS. 18A to 23C, and kit of parts including any one of device 10R to 10W and an applicator and/or effector, adapted to any one of device 10R to 10W. Method 3800 includes: (i.) attaching the device to the fastener of the applicator (step 3802); (ii.) identifying an attachment area on the tissue for repair (step 3804); (iii.) positioning the applicator holding the device at the attachment area by positioning the attachment area between the fastener and the positioning area of the applicator, the positioning area having a suture passer with a suture threaded through a needle arranged on said suture passer (step 3806); (iv.) applying the force on the fastener of the applicator holding the device on the attachment area to actuate the needle through the tissue and to secure the device to the tissue for repair (step 3808); and (v.) releasing the fastener of the applicator from the attachment area, leaving the device secured to the tissue for repair (step 3810).

In step 3808 of method 3800, applying the force on the fastener of the applicator holding the device on the attachment area to actuate the needle through the tissue and to secure the device to the tissue for repair comprises: (a.) releasing the needle from the suture passer such that the needle moves towards an aperture arranged on the fastener; and (b.) detaching the device from the fastener of the applicator. Alternatively, in some embodiments, step 3808 of method 3800 may instead include (c.) detaching the device from the fastener of the applicator; and (d.) releasing the needle from the suture passer such that the needle moves towards an aperture arranged on the fastener. In various embodiments, releasing the needle from the suture passer such that the needle moves towards the aperture arranged on the fastener includes piercing the tissue to deliver the suture as the needle moves from the positioning area to the fastener. In various embodiments, the aperture on the fastener may be in alignment with the aperture of the device.

In some embodiments of method 3800, the suture may be threaded through the device before releasing the fastener of the applicator from the attachment area. In some other embodiments of method 3800, the suture may be threaded through the device after releasing the fastener of the applicator from the attachment area.

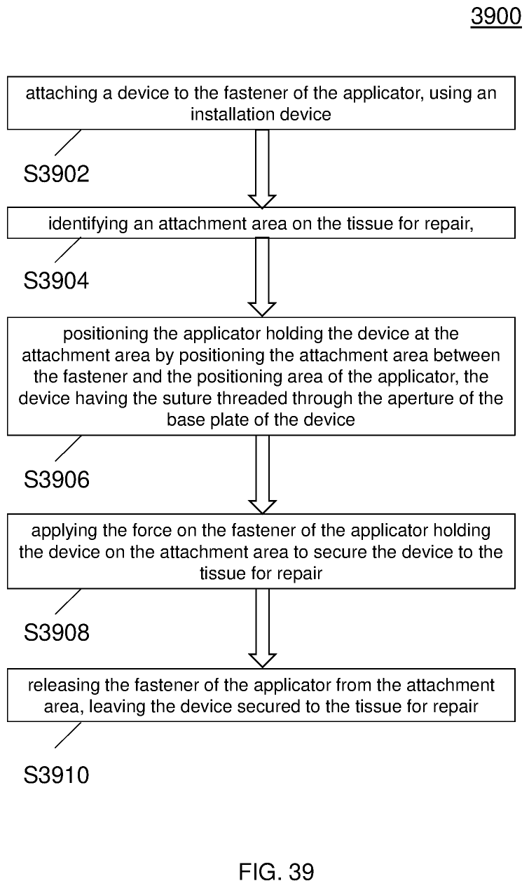

FIG. 39 shows another embodiment of method 3900 of repairing a tissue, according to another aspect of the disclosure. The steps of method 3900 may be based on any one of device 10T to 10AB as described with reference to FIGS. 20A to 27C, and kit of parts including any one of device 10T to 10AB, an applicator including an effector adapted to any one of device 10T to 10AB, and an installation device adapted to any one of device 10T to 10AB. The method of attaching any one of device 10T to 10AB to the applicator, using the installation device has been described with reference to method 3500 and FIGS. 35 to 361.

Method 3900 includes: (i.) attaching the device to the fastener of the applicator, using the installation device (step 3902); (ii.) identifying an attachment area on the tissue for repair (step 3904); (iii.) positioning the applicator holding the device at the attachment area by positioning the attachment area between the fastener and the positioning area of the applicator, the device having the suture threaded through the aperture of the base plate of the device (step 3906); (iv.) applying the force on the fastener of the applicator holding the device on the attachment area to secure the device to the tissue for repair (step 3908); and (v.) releasing the fastener of the applicator from the attachment area, leaving the device secured to the tissue for repair (step 3910).

In step 3706 of method 3700, or step 2806 of method 3800, or step 3906 of method 3900, positioning the attachment area between the fastener and the positioning area of the applicator may include positioning the applicator such that the tissue for repair rests on the positioning area, and the fastener holding the device is above the tissue for repair.

In step 3708 of method 3700, or step 3808 of method 2900, or step 3908 of method 3900, applying the force on the fastener of the applicator holding the device on the attachment area to secure the device to the tissue for repair may include applying the force on the fastener in a fixed movement towards the positioning area of the applicator to push the device into the tissue for repair, wherein the positioning area provides a counter support when the fastener is pushed against the tissue for repair, and wherein the plurality of anchor members of the device is configured to be positioned in the plurality of holes on the positioning area.

Accordingly, upon release of the fastener of the applicator (in step 3710 or 3810 or 3910), the device may be secured to the tissue for repair by the plurality of anchor members which penetrate the tissue for repair, and wherein the base plate rests on the tissue for repair. For example, the first surface of the base plate may rest on reattached or repaired tissue.

FIGS. 40A to 40C shows schematic illustrations showing the conventional repair of torn tissue with and without the device 4000. The device 4000 may refer to device 10A to 10AB described with reference to FIGS. 1A to 27C, for surgical repair of the rotator cuff tissue. FIG. 40A shows the torn tissue repaired by conventional suture configurations; and FIGS. 40B and 40C show the torn tissue repaired by conventional suture configurations and further reinforced by the device 4000. As shown in FIGS. 40B and 40C, the device may be positioned at various end points along the suture configuration, and be positioned to overlay the torn tissue. In various embodiments, the device 4000 may be compatible with existing suture configurations for tissue, e.g. rotator cuff, repairs. In particular, the device 4000 may be compatible with the single-row repair (FIG. 40B), and the suture-bridge (FIG. 40C) suture configurations.

The device in accordance with various embodiments of the disclosure may be positioned at typical suture-tissue interfaces to minimize direct contact between the suture and the torn tissue along the lateral plane of the torn tissue to minimize or reduce re-tearing of the repaired tissue, and to redistribute mechanical tension or stress typically borne at the suture-tissue interface. For example, a point stress may be re-distributed to various points on the tissue, via the plurality of anchor members and/or the central shaft of the device. In addition, the plurality of anchor members of the device which are spaced apart from each other, and in some embodiments, spaced apart from the central shaft, enables the individual adjustment of each anchor member and/or the central shaft of the device (which may be dependent on the size or severity of the tissue tear), and personalization of the device. As a result, a personalized and customized device which reduces or minimizes the detachment of the tissue from bone due to repeated tension or stress, and consequently reduces the need for surgical re-attachment of the detached tissue to bone, may be provided.

According to another aspect of the disclosure there is a method of manufacturing the device as described, comprising the steps of: providing a base plate having a first surface and a second surface opposing the first surface; forming an aperture on a portion of the base plate, the aperture configured to receive a suture; providing a plurality of anchor members extending from the first surface of the base plate, each of the plurality of anchor members having an end configured to penetrate the tissue, wherein each of the plurality of anchor members is spaced apart from another anchor member.

In some embodiments, the method of manufacture comprises a step of integrally molding the connection between the anchor members 130, the first segment 116, and the second segment 117 as a single body. In some embodiments, the method of manufacture comprises a step of integrally molding the first bar 2502 and second bar 2504 as a single body.

In some embodiments, the method of manufacture will include machining out an implant from a raw material bar. The machining out may be carried out by a CNC process. In some embodiments, the integrally molding step may include injection molding, the injection molding may comprise at least one of the following: a standard injection molding, a compression molding, a micro injection molding, an over-molding. In the over-molding step, two materials may be used for the implant, a metallic skeleton may be used to provide strength and hardness (i,e. sharpness at the anchor) and then the “skeleton” is over-molded with plastic to complete the rest of the body. Over-molding may be useful to minimize/reduce the amount of metal used in the manufacturing.

In some embodiments, additional sharpening process might be needed to sharpening the anchor members 130.

While the disclosure has been particularly shown and described with reference to specific embodiments, it should be understood by those skilled in the art that various changes in form and detail may be made therein without departing from the spirit and scope of the disclosure as defined by the appended claims of the invention. The scope of the disclosure is thus indicated by the appended claims and all changes which come within the meaning and range of equivalency of the claims are therefore intended to be embraced.

1. A device for tissue repair comprising
a base plate having a first surface and a second surface opposing the first surface;
an aperture arranged on a portion of the base plate, the aperture configured to receive a suture;
a plurality of anchor members extending from the first surface of the base plate, each of the plurality of anchor members having an end configured to penetrate the tissue,
wherein each of the plurality of anchor members is spaced apart from another anchor member.

2. The device of claim 1, further comprising a central shaft extending from the first surface of the base plate, the central shaft having the aperture arranged on the portion of the base plate,
wherein a length of the central shaft is shorter than, same as, or longer than a length of each of the plurality of anchor members;
wherein the central shaft comprises a partial circle, or a full circle;
wherein the central shaft is pivotable with respect to the portion of the base plate; and/or
wherein a surface area of the central shaft is greater than a surface area of the suture, along a longitudinal axis of the device.

3. (canceled)

4. (canceled)

5. (canceled)

6. The device of claim 1, wherein the plurality of anchor members each comprise a rectangular frame extending from the base plate, wherein a length of the rectangular frame forms the end configured to penetrate the tissue, optionally, further comprising, two rectangular frames joining each other at a breath to form a V-shape; and/or
wherein the plurality of anchor members each comprise, a first portion extending from the first surface of the base plate, a second portion extending from an end of the first portion and towards a central axis of the base plate.

7. (canceled)

8. (canceled)

9. The device of claim 1, wherein the plurality of anchor members each comprise an elongate portion extending perpendicular from the base plate, or at an angle to the base plate.

10. The device of claim 9, further comprising a set of teeth arranged along a length of the elongate portion;
wherein the elongate portion comprises a pointed tip which forms the end configured to penetrate the tissue;
wherein the elongate portion comprises a beveled tip which forms the end configured to penetrate the tissue;
wherein the elongate portion comprises an arrowhead tip which forms the end configured to penetrate the tissue, optionally, further comprising, a first part having a first thickness, and a second part positioned adjacent to the first part and proximal to the arrowhead tip of the shaft, the second part having a second thickness greater than the first thickness; or
wherein the elongate portion comprises a first fluke which forms the end configured to penetrate the tissue, optionally, further comprising a second fluke arranged along the length of the elongate portion;
wherein the elongate portion further comprises, a sickle-head tip which forms the end configured to penetrate the tissue, a third fluke arranged on an end of the sickle-head tip, optionally, further comprising a fourth fluke arranged along the length of the elongate portion;
wherein the elongate portion is configured to move towards a central axis of the base plate in response to a force applied on the length of the elongate portion, and to move away from the central axis of the base plate in response to release of the force; or
wherein an end of the elongate portion is configured to crimp towards a central axis of the base plate, in response to a force applied against the end of the elongate portion.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The device of claim 1, wherein the plurality of anchor members are arranged along a perimeter of the base plate, or wherein the plurality of anchor members are arranged to be spaced apart from a perimeter of the base plate; and/or
wherein the base plate comprises a partial circle, or a full circle; or wherein the base plate comprises, a first segment, a second segment, and a third segment, wherein the first, second and third segments are connected to each other at the portion of the base plate having the aperture; and/or
wherein the base plate further comprises a plurality of through-holes configured to receive the plurality of anchor members, and wherein the plurality of anchor members are configured to be attachable to the plurality of through-holes on the base plate; and/or
wherein the base plate further comprises one or more mounting brackets extending from the second surface of the base plate.

23. (canceled)

24. (canceled)

25. (canceled)

26. The device of claim 22,
wherein the first segment and the second segment is spaced apart by a first angle, wherein the second segment and the third segment is spaced apart by a second angle, wherein the first segment and the third segment is spaced apart by the second angle, wherein the first and second segments are identical to each other, and wherein the first angle differs to the second angle; or wherein the first segment and the second segment is spaced apart by a third angle, wherein the second segment and the third segment is spaced apart by the third angle, wherein the first segment and the third segment is spaced apart by the third angle, and wherein the first, second and third segments are identical to each other; and/or
wherein the third segment comprises, a first bar extending from the portion of the base plate having the aperture, a second bar extending from an end of the first bar in a direction perpendicular to the first bar, and wherein the second bar comprises a slot arranged on a first surface of the second bar;
wherein the third segment is pivotable with respect to the first and second segments of the base plate; and/or
wherein the device further comprises, a gate arranged to connect an end of the first segment to an end of the second segment, optionally, the gate being configured to extend towards an interface where the first and second segments are connected and comprising a knob positioned at the interface where the first and second segments are connected.

27. (canceled)

28. (canceled)

29. (canceled)

30. (canceled)

31. (canceled)

32. (canceled)

33. (canceled)

34. (canceled)

35. A kit of parts of a tissue repair assembly comprising
a device for tissue repair comprising
a base plate having a first surface and a second surface opposing the first surface;
an aperture arranged on a portion of the base plate, the aperture configured to receive a suture;
a plurality of anchor members extending from the first surface of the base plate, each of the plurality of anchor members having an end configured to penetrate the tissue,
wherein each of the plurality of anchor members is spaced apart from another anchor member.

36. The kit of claim 35, further comprising an applicator comprising
a fastener, the fastener having an effector arranged along a length of the fastener, the effector dimensioned and sized to hold the base plate of the device;
a platform arranged opposite the fastener, the platform having a positioning area comprising a plurality of holes for receiving the plurality of anchor members of the device;
wherein the fastener is connected along a length of the platform via a first pivotable interface,
wherein an application of a force on the fastener is configured to cause the fastener to move towards the positioning area.

37. The kit of claim 36, wherein the effector comprises
a first beam, a second beam and a third beam, the first and second beams extending from a part of the third beam, wherein the first beam and the second beam are each configured to hold a first segment and a second segment of the base plate, wherein the first beam, the second beam and the third beam are dimensioned to form an opening, the opening configured to receive a third segment of the base plate, and wherein the application of the force on the fastener is configured to cause the effector to move towards an end of the fastener to hold the first, second and third segments of the base plate of the device; or
a first plate configured to hold a third segment of the base plate; a first clasp arranged on the first plate, the first clasp configured to slide along the length of the fastener or part thereof, wherein the application of the force on the fastener is configured to cause the first clasp to move towards the first pivotable interface, and to cause the first plate to move towards an end of the fastener to hold the third segment of the base plate of the device; or
a second plate having a protrusion dimensioned to receive a slot arranged on a third segment of the base plate; a second clasp arranged on the second plate, the second clasp configured to slide along the length of the fastener or part thereof, wherein the application of the force on the fastener is configured to cause the second clasp to move towards the first pivotable interface, and to cause the second plate to move towards an end of the fastener to hold the slot arranged on the third segment of the base plate of the device.

38. (canceled)

39. (canceled)

40. (canceled)

41. The kit of claim 40, wherein the first module of the installation device further comprises a second component arranged opposite the first component, the second component connected to the first component via a second pivotable interface, wherein the second pivotable interface is configured to fit within the first pivotable interface connecting the fastener along the length of the platform; and/or
wherein the second module of the installation device further comprises, a wire loop connected to a part of the second module, the wire loop configured to receive the suture; a hole arranged on the second module, the hole configured to receive the wire loop and the suture, wherein the hole on the second module is configured to be in alignment with the aperture of the base plate of the device, and wherein the second module is configured to be removable from the first component of the first module, such that the wire loop and the suture threads through the hole of the second module.

42. (canceled)

43. (canceled)

44. (canceled)

45. (canceled)

46. A method of repairing a tissue comprising
attaching the device according to claim 2 to the fastener of the applicator according to claim 36;
identifying an attachment area on the tissue for repair, the attachment area having a suture passing through the tissue for repair;
positioning the applicator holding the device at the attachment area, wherein positioning the applicator comprises
positioning the attachment area between the fastener and the positioning area of the applicator;
positioning the aperture of the central shaft to be in contact with the suture passing through the tissue for repair;
applying the force on the fastener of the applicator holding the device on the attachment area to secure the device to the tissue for repair;
releasing the fastener of the applicator from the attachment area, leaving the device secured to the tissue for repair.

47. A method of repairing a tissue comprising
attaching the device according to claim 1 to the fastener of the applicator according to claim 36;
identifying an attachment area on the tissue for repair;
positioning the applicator holding the device at the attachment area by positioning the attachment area between the fastener and the positioning area of the applicator, the positioning area having a suture passer with a suture threaded through a needle arranged on said suture passer;
applying the force on the fastener of the applicator holding the device on the attachment area to actuate the needle through the tissue and to secure the device to the tissue for repair; and
releasing the fastener of the applicator from the attachment area, leaving the device secured to the tissue for repair.

48. (canceled)

49. (canceled)

50. A method of repairing a tissue comprising
attaching the device according to claim 1 to the fastener of the applicator according to claim 36, using the installation device according to claim 40;
identifying an attachment area on the tissue for repair;
positioning the applicator holding the device at the attachment area by positioning the attachment area between the fastener and the positioning area of the applicator, the device having the suture threaded through the aperture of the base plate of the device;
applying the force on the fastener of the applicator holding the device on the attachment area to secure the device to the tissue for repair; and
releasing the fastener of the applicator from the attachment area, leaving the device secured to the tissue for repair.

51. (canceled)

52. (canceled)

53. (canceled)

54. (canceled)

55. A method of manufacturing a device for tissue repair, comprising the steps of:
providing a base plate having a first surface and a second surface opposing the first surface;
forming an aperture on a portion of the base plate, the aperture configured to receive a suture;
providing a plurality of anchor members extending from the first surface of the base plate, each of the plurality of anchor members having an end configured to penetrate the tissue,
wherein each of the plurality of anchor members is spaced apart from another anchor member.

56. The method of claim 58, comprising the step of integrally molding the connection between the anchor members, the first segment, and the second segment as a single body.

57. The method of claim 59, comprising the step of integrally molding the first bar and second bar as a single body.

58. The method of claim 55, wherein the base plate comprises, a first segment, a second segment, and a third segment; wherein the first, second and third segments are connected to each other at the portion of the base plate having the aperture.

59. The method of claim 58, wherein the third segment comprises, a first bar extending from the portion of the base plate having the aperture, a second bar extending from an end of the first bar in a direction perpendicular to the first bar, and wherein the second bar comprises a slot arranged on a first surface of the second bar.
